(19) United States
(12) Patent Application Publication
(10) Pub. No.: US 2008/0050372 A1
(43) Pub. Date: Feb. 28, 2008

(54) INHIBITION OF ALPHA-2 HS GLYCOPROTEIN (AHSG/FETUIN) IN OBESITY AND INSULIN CONTROL OF GLUCOSE HOMEOSTASIS

(75) Inventors: George Grunberger, Grosse Pointe Park, MI (US); Suresh T. Mathews, Rockville, MI (US); Kai-Lin Catherine Jen, Bloomfield Hills, MI (US); Anton Scott Goustin, Ferndale, MI (US); Pothur R. Srinivas, Laurel, MD (US)

Correspondence Address: BROWDY AND NEIMARK, P.L.L.C.;624 NINTH STREET, NW, SUITE 300, WASHINGTON, DC, 20001-5303, US

(73) Assignee: Wayne State University, Detroit, MI (US)

(21) Appl. No.: 11/773,883

(22) Filed: Jul. 5, 2007

Related U.S. Application Data
(63) Continuation of application No. 10/415,288, filed on Mar. 16, 2004, now abandoned.
(60) Provisional application No. 60/243,442, filed on Oct. 27, 2000.

(51) Int. Cl. A61K 48/00; A61K 38/46; A61P 3/00; A61P 3/04; A61K 39/395

(52) U.S. Cl. 424/133.1; 424/130.1; 424/141.1; 424/94.6; 514/44

(57) ABSTRACT

α2-Heremans Schmid Glycoprotein (AHSG) inhibits insulin-induced autophosphorylation of the insulin receptor (IR) and IR-tyroskine kinase (TK) activity; genetic ablation of the Ahsg gene enhances insulin signal transduction and increase whole-body insulin sensitivity. Therefor, AHSG and its gene(s) are useful targets for agents that inhibit the development or progression of Type II diabetes or any disease or disorder associated with increased insulin resistance. Provided herein is a method for inhibiting the biological activity of AHSG protein in a cell using compounds that inhibit phosphorylation of AHSG. Also disclosed is a method of augmenting the phosphorylation or IR-TK activity in a liver or muscle cell by providing a compound that lowers the amount of active AHSG or inhibits the biological activity of AHSG. Such effects may be achieved by delivering an antisense nucleic acid construct that hybridizes with AHSG encoding DNA. This invention includes a method (a) treating a subject that is susceptible to, or suffers from, obesity and insulin resistance or (b) increasing insulin sensitivity, and thereby preventing or treating insulin resistance in the subject. The method comprises lowering the amount of active AHSG or inhibiting the biological activity of AHSG in the subject, preferably in liver or muscle, by using AHSG antisense constructs or an anti-AHSG antibody. In a subject eating a high fat diet, the effect on body weight gain and/or insulin resistance is diminished, and total body fat content is lowered, by lowering the amount of active AHSG or inhibiting the action of the AHSG in the subject using the agents noted above.

### BACKGROUND OF THE INVENTION

1. Field of the Invention

The present invention is directed to new functions of the plasma glycoprotein α2-Heremans Schmid Glycoprotein (fetuin) leading to novel approaches to the treatment of obesity and to regulation of insulin control of glucose homeostasis.

2. Description of the Background Art

Insulin controls glucose homeostasis by stimulating the clearance of glucose into skeletal muscle, liver and adipose tissue. Diabetes mellitus is a group of metabolic disorders characterized by elevated levels of glucose. This results from a defect in secretion of insulin or insulin action or both. Insulin resistance, defined as an attenuated response to physiological or supraphysiologicial levels of insulin, is shared by common pathological conditions such as obesity, hypertension, dyslipidemia, glucose intolerance, pregnancy and type 2 diabetes mellitus.

Insulin exerts its effects by binding to its receptor, which activates a tyrosine kinase enzymatic activity, inherent to the receptor. The phosphorylating action of this protein sets into motion a cascade of signaling events leading to uptake of glucose into muscle and adipocytes.

Based on information of worldwide prevalence, type 2 diabetes is considered to have reached epidemic proportions (King, H. & Rewers, Diabetes Care 16, 157-177 (1993)). Parallel to the rise in type 2 diabetes, is a rapid expansion of obesity, especially in westernized societies where the condition is associated with consumption of a high fat (HF) diet (Hill, J. O. et al., J Nutr 130 (suppl.2), 284S-288S (2000)). Insulin resistance, characterized by varying levels of attenuated response to physiological and supra-physiological levels of insulin, is central to the pathophysiology of obesity and type 2 diabetes (Reaven, G. M., Diabetes 37, 1595-1607 (1988)). At the cellular level, insulin resistance is characterized by insulin receptor (IR) down-regulation, reduced IR kinase activity and/or defects in the intracellular signaling responses to insulin (Thies, R. S. et al., Diabetes 39, 250-259 (1990); Saad, M. J. A. et al., J Clin Invest 90, 1839-1849 (1992); Heydrick, S. J. et al., J Clin Invest 91, 1358-1366 (1993); Le Marchand-Brustel, Y. Exp Clin Endocrinol Diabetes 107, 126-132 (1999)).

Several physiological modulators of IR function, involved in the pathogenesis of insulin resistance, have been described, and include TNF-α, PC-1, Rad, protein tyrosine phosphatases and the plasma glycoprotein α2-Heremans Schmid Glycoprotein (abbreviated α2-HSG or AHSG) which is a member of the fetuin family and has therefore also been referred to as fetuin. (Moller, D. E., Trends Endocrinol Metab 11, 212-217 (2000); Goldfine, I. D. et al., Ann NY Acad Sci 892, 204-222 (1999); Reynet, C. et al., Science 262, 1441-1444 (1993); Ahmad, F. et al., J Clin Invest 100, 449-458 (1997); Srinivas, P. R. et al., Mol Endocrinol 7, 1445-55 (1993)).

Nomenclature of the AHSG s protein is still not standardized as the human and murine proteins are typically termed α₂-HS-glycoprotein or AHSG whereas the rat and bovine protein is more often termed “fetuin.” The name “AHSG” will be used herein to refer to this protein in any mammalian species. The gene encoding AHSG will be designated herein as Ahsg. α2-HS glycoprotein (AHSG), a glycoprotein present in the serum, is synthesized by hepatocytes. The AHSG molecule consists of two polypeptide chains, which are both cleaved from a proprotein encoded from a single mRNA. It is known to be involved in several functions, such as endocytosis, brain development and the formation of bone tissue. The protein is commonly present in the cortical plate of the immature cerebral cortex and bone marrow hemopoietic matrix, and it has therefore been postulated that it participates in the development of the tissues. However, prior to the work of the present inventors and their colleagues, and to the making of the present invention, its exact significance has been largely obscure.

AHSG is a natural inhibitor of the insulin-stimulated IR tyrosine kinase (IR-TK) (Srinivas, P. R. et al., Mol Endocrinol 7, 1445-55 (1993); Auberger, P. et al., Cell 58, 631-640 (1989); Rauth, G. et al., Eur. J. Biochem 204, 523-529. (1992); Haasemann, M. et al., Biochem J 274, 899-902 (1991); Srinivas, P. R. et al., Biochem Biophys Res Commun 208, 879-85 (1995); Kalabay, L. et al., Horm Metab Res 30, 1-6 (1998)).

The phosphorylation status of AHSG is of critical importance for TK inhibition (Auberger, P. et al., supra; Akhoundi, C. et al., J Biol Chem 269, 15925-15930 (1994)). Nearly 20% of the circulating AHSG pool is phosphorylated on Ser-120 and Ser-312 to approx. 1 mol of phosphate/mol of protein (Haglund, A. C. et al., Biochem J 357, 437-445 (2001)). AHSG inhibits IR-TK by reducing the Vₘₐₓ of the insulin-stimulated IR-TK reaction and increasing the S_{0.5} for ATP and for polyGT (Grunberger, G. et al., in Frontiers in Animal Diabetes Research: Insulin Signaling. From Cultured Cells to Animal Models, Vol. 3 (eds. Grunberger, G. & Zick, Y.) (Harwood Academic Publishers, 2001)).

AHSG preferentially interacts with the activated IR and does not require the proximal 576 amino acids of IR α-subunit for its IR autophosphorylation or its TK inhibitory activity (Mathews, S. T. et al., Mol Cell Endrocrinol 264, 87-98 (2000)).

Acute injection of human recombinant AHSG inhibits insulin-stimulated tyrosine phosphorylation of IR β-subunit and IRS-1, in rat liver and skeletal muscle.

Ahsg gene expression is significantly increased in a rat model of diet-induced obesity, (Lin, X. et al., Life Sci 63, 145-153 (1998)). Evidence of IR-TK inhibitory function of human bovine, mouse, sheep and pig AHSG suggests a conserved function for AHSG or fetuin homologs (Srinivas et al., 1993, supra; Grunberger, G. et al., supra; Mathews, S. T. et al., Life Sci 61, 1583-92 (1997); Cintrón, V. J. et al., Int J Exp Diab Res 1, 249-263 (2001)).

The human Ahsg gene resides on chromosome 3q27, which has been recently mapped as a type 2 diabetes-susceptibility locus (Vionnet, N. et al., Am J Hum Genet 67, 1470-1480 (2000)). Kissebah et al. have demonstrated a quantitative trait locus on chromosome 3q27 strongly linked to the metabolic syndrome (Kissebah, A. H. et al., Proc Natl Acad Sci USA 97, 14478-14483 (2000)). Mice with a targeted deletion of Ahsg are fertile and demonstrate no gross anatomical abnormalities except for the presence of ectopic microcalcifications in a minority of retired female breeders (Jahnen-Dechent, W. et al., J Biol Chem 272, 31496-31503 (1997)). In humans, no complete AHSG deficiency has been found in extensive population studies and clinical investigations (Osawa, M. et al., Ann Hum Genet 65, 27-34 (2001)).

Citation of the above documents is not intended as an admission that any of the foregoing is pertinent prior art. All statements as to the date or representation as to the contents of these documents is based on the information available to the applicant and does not constitute any admission as to the correctness of the dates or contents of these documents.

### SUMMARY OF THE INVENTION

To clarify the role of AHSG in insulin action, the present inventors explored glucose homeostasis in mice carrying two null alleles for Ahsg. Since AHSG inhibits insulin induced IR-autophosphorylation and TK activity, it was hypothesized that genetic ablation of AHSG results in enhanced insulin signal transduction and increased whole-body insulin sensitivity. Further, the consequence of this genetic manipulation was examined in a model of acquired insulin resistance, HF feeding. The present inventors and their colleagues discovered that human, murine and bovine AHSG inhibits insulin-stimulated IR autophosphorylation and TK activity in vitro, in intact cells or when injected into a mammalian s subject.

Because the Ahsg gene is located on human chromosome 3q27 (and its ortholog in mouse maps to the syntenic mouse chromosome 16), recently identified as a susceptibility locus for type 2 diabetes and the metabolic syndrome, the present inventors explored insulin signaling, glucose homeostasis and the effect of feeding a HF diet on weight gain, body fat composition and glucose disposal in mice carrying two null alleles for Ahsg (B6. 129-Ahsg^{tm1Mb1}) Knockout (KO) mice demonstrated increased basal and insulin-stimulated phosphorylation of IR and downstream signaling molecules, MAP kinase and the Ser-Thr kinase Akt in liver and skeletal muscle of the KO mice. Glucose and insulin tolerance tests in Ahsg KO mice indicate significantly enhanced glucose clearance and insulin sensitivity. Ahsg KO mice show normal fasting blood glucose and insulin levels. Ahsg KO mice subjected to euglycemic-hyperinsulinemic clamp show augmented sensitivity to insulin evidenced by increased glucose infusion rate and significantly increased skeletal muscle glycogen content. When fed a high-fat diet, Ahsg KO mice were resistant to weight gain, demonstrate decreased body fat and remained insulin sensitive. In contrast, wild-type (WT) mice fed a HF diet showed increased levels of insulin and decreased insulin sensitivity. These results suggest to the present inventors that AHSG plays a critical role in regulating postprandial glucose disposal, insulin sensitivity, weight gain and fat accumulation and presents a novel therapeutic target in the treatment of type 2 diabetes, obesity and other insulin resistant conditions.

Based on the following observations, the present inventors conceived that feeding a high-fat diet to Ahsg-null mice would not result in body weight-gain:
1. Visual examination of age and sex-matched female mice demonstrated lesser fat depots (white fat) in Ahsg-null mice.
2. Ahsg-null mice had significantly lower amounts of free fatty acids.
3. Serum triglyceride levels were significantly lower in Ahsg-null mice

Since AHSG inhibits insulin-induced IR autophosphorylation and IR-TK activity, the present inventors conceived that that genetic ablation of the Ahsg gene would result in enhanced insulin signal transduction and increase whole-body insulin sensitivity. Several lines of evidence described herein indicate Ahsg knockout mice have increased glucose clearance and insulin sensitivity. This makes AHSG and its gene(s) useful targets for developing agents that inhibit the development or progression of Type II Diabetes or any disease or disorder associated with increased insulin resistance.

The present invention provides a method for inhibiting the biological activity of AHSG protein in a cell comprising providing to the cell a compound that inhibits the phosphorylation of AHSG at one or both of Ser-120 and Ser-312 or dephosphorylates one or both of Ser-120 and Ser-312. Preferably, the biological activity comprises the binding of AHSG to muscle IR or the diminution of IR function. The above inhibiting may be achieved by contacting the cell with a protein serine-threonine kinase inhibitor, a serine phosphatase or a compound that induces or enhances the activity of the phosphatase, or a combination of both types of agents.

Also provided is a method of augmenting the phosphorylation or tyrosine kinase activity of insulin receptors in a liver or muscle cell, comprising providing to the cell a compound that lowers the amount of active AHSG or inhibits the biological activity of AHSG in the cell, thereby augmenting the phosphorylation and/or the tyrosine kinase activity.

The above augmenting is achieved by delivering to the cell an effective amount of an antisense nucleic acid construct that hybridizes with a sequence present in AHSG genomic DNA or with a coding nucleic acid sequence that encodes AHSG, thereby lowering the amount or inhibiting the activity of AHSG in the subject. The genomic DNA preferably has the sequence SEQ ID NO:1. The above coding sequence preferably encodes a protein having a sequence selected from the group consisting of SEQ ID NO:2, SEQ ID NO:3, SEQ ID NO:5, SEQ ID NO:6 or SEQ ID NO:7. In the above method, the compound may be one that inhibits the phosphorylation of AHSG at one or both of Ser-120 and Ser-312 or dephosphorylates one or both of Ser-120 and Ser-312.

In another embodiment, the invention is directed to a method for treating a subject that is susceptible to, or suffers from, obesity and insulin resistance comprising lowering the amount of active AHSG or inhibiting the biological activity of AHSG in the subject. The lowering or the inhibiting is preferably in liver or muscle. The inhibiting may be achieved by delivering to the subject an effective amount of an antisense nucleic acid construct that hybridizes with a sequence present in AHSG genomic DNA or with a coding nucleic acid sequence that encodes AHSG, thereby lowering the amount or inhibiting the activity of AHSG in the subject. In the above method of the genomic DNA preferably has the sequence SEQ ID NO:1. The antisense nucleic acid preferably has between about 6 and about 30 nucleotides. The antisense construct may be is antisense to a sequence that includes the initiation codon of the AHSG. In another embodiment, the antisense construct is antisense to a sequence that is part or all of an intron of SEQ ID NO:1.

The above coding sequence encodes a protein preferably has a sequence selected from the group consisting of SEQ ID NO:2, SEQ ID NO:3, SEQ ID NO:5, SEQ ID NO:6 or SEQ ID NO:7, most preferably SEQ ID NO:2 or SEQ ID NO:3. The inhibiting is also achieved by administering to the subject an effective amount of an antibody specific for an epitope of AHSG, whereby the antibody lowers the amount of inhibits the biological activity of AHSG.

The antibody is preferably a monoclonal antibody; most preferably the subject is a human and the antibody is human or a humanized antibody.

Also provided is a method for increasing insulin sensitivity, and thereby preventing or treating insolent resistance, a in subject in need thereof comprising lowering the amount of active AHSG or inhibiting the action of the in the subject.

Another method is directed to treating a condition associated with decreased action of insulin in peripheral tissues of a subject, comprising lowering the amount of active AHSG or inhibiting the biological activity of AHSG in the subject.

The invention includes a method for preventing or diminishing the effect of a high-fat diet on body weight gain and/or insulin resistance in a subject eating a high fat diet, comprising lowering the amount of active AHSG or inhibiting the action of the AHSG in the subject.

Also provided is a method of lowering total body fat content in a subject eating a high fat diet comprising lowering the amount of active AHSG or inhibiting the action of the AHSG in the subject.

### BRIEF DESCRIPTION OF THE DRAWINGS

FIGS. 1-4 show IR autophosphorylation and TK activity in wildtype as compared to KO mice. FIG. 1 shows in vitro IR autophosphorylation. FIG. 2 shows IR-TK activity. FIG. 3 shows liver IR autophosphorylation and TK activity. FIG. 4 shows muscle IR autophosphorylation and TK activity. In FIGS. 1 and 2, results were determined from IRs partially purified from liver membrane material fractionated on wheat-germ agglutinin. A representative autoradiograph (of 4 separate experiments with IRs purified individually from livers of weight-matched, 8-10 week-old male WT and KO mice, n=4 per genotype) of in vitro IR-β subunit autophosphorylation (basal or in the presence of 1, 10 or 100 nM insulin) is illustrated in the upper panel of FIG. 1. A Western blot of IR-β subunit confirming equal loading of IR (FIG. 1, lower panel). The combined data of 4 separate experiments is represented in the bar graph of FIG. 1.

FIGS. 3 and 4: To assess the status of in vivo basal and insulin-induced IR autophosphorylation, saline or insulin (0.1, 1, or 10 μM) was injected through the portal vein of weight-matched, 8-10 week old male WT and KO mice. IR was immunoprecipitated from liver (FIG. 3) or muscle (FIG. 4) homogenates with an anti-IR-β subunit antibody and immunoblotted with an anti-phosphotyrosine antibody. Samples were normalized for loading by assaying total level of IR-β subunit. The quantified data (ratio of IR autophosphorylation to total level of IR β-subunit) are shown as bar graphs in FIGS. 3 and 4 diagrams (mean±S.E.M. of n=4 mice per genotype). *p<0.05, ** p<0.01, *** p<0.001. WT vs. KO

FIG. 5 and FIG. 6 show results measuring insulin signal transduction. Weight-matched, 8-10 week old male WT and KO mice were studied.

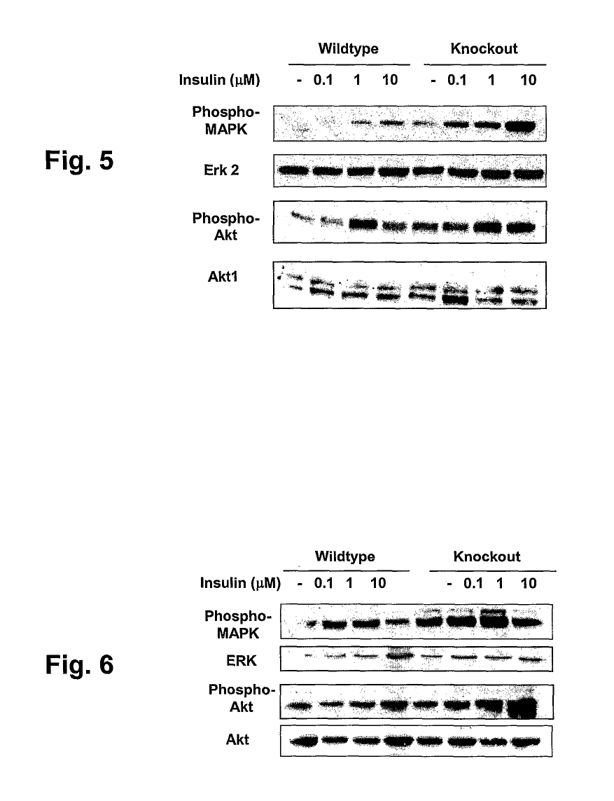

In the experiment for FIG. 5, liver homogenates from mice injected with saline- or insulin (0.1, 1, or 10 μM) were resolved on SDS-PAGE, transferred and detected by chemiluminescence with antibodies against phospho-MAPK (panel 1), or phospho-Akt (panel 3). Membranes were stripped and blotted for ERK2 (panel 2) and Akt1 (panel 4) respectively, to normalize for sample loading. A representative blot (from 4-5 separate experiments) for each protein is presented.

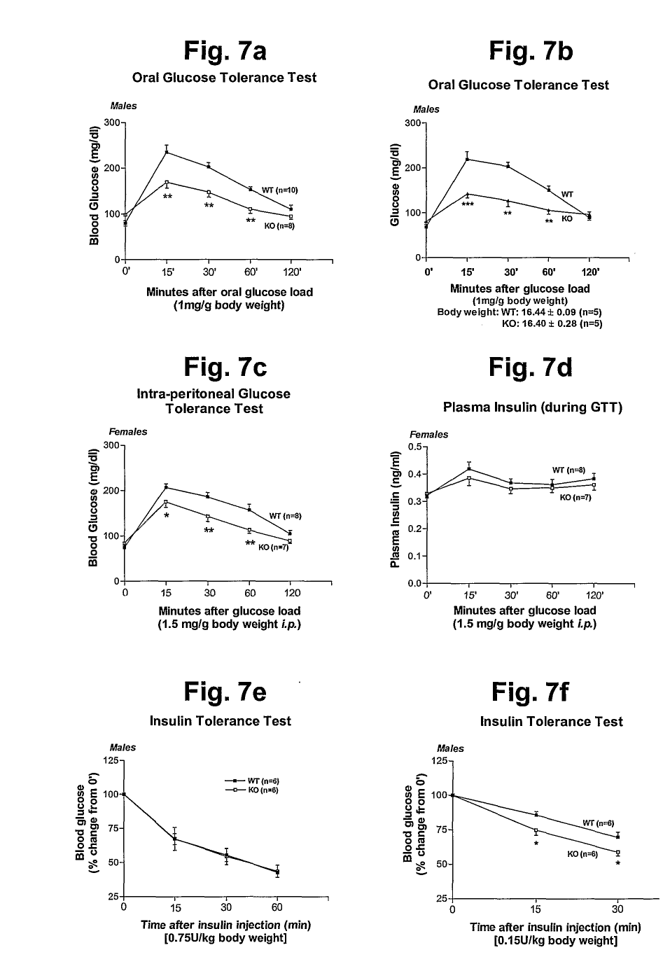

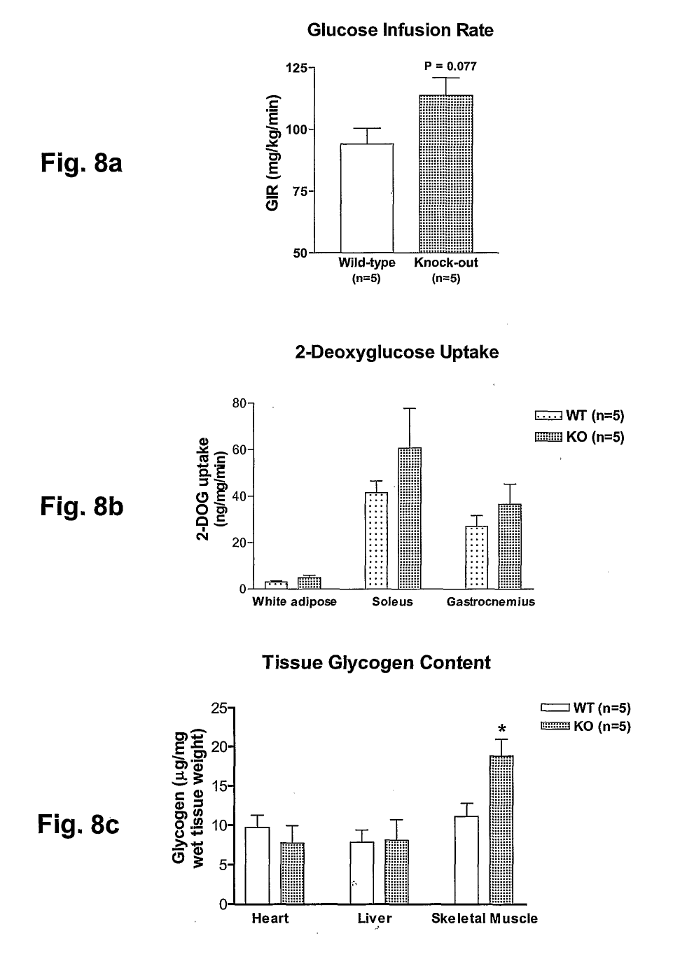

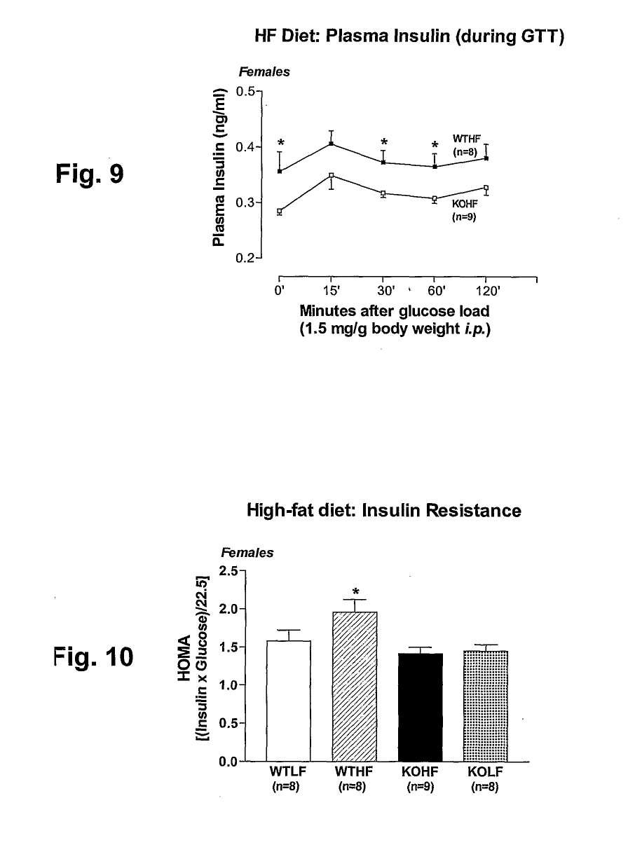

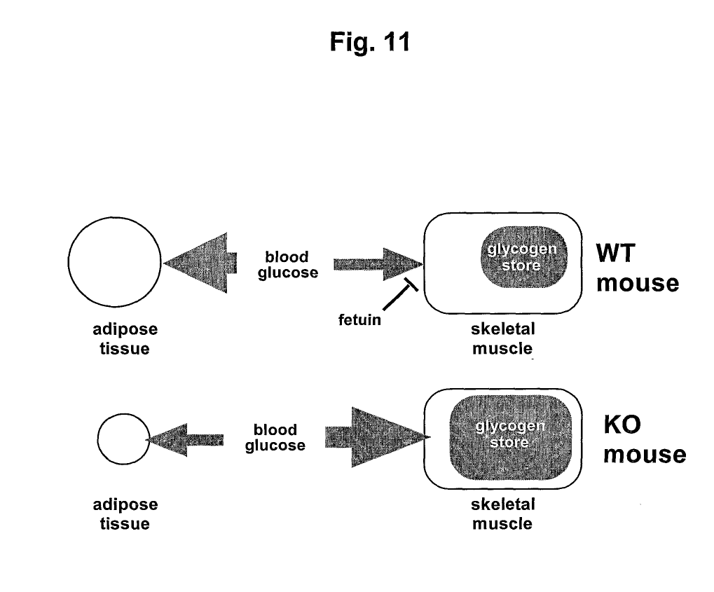

In the experiments for FIG. 6, hindlimb muscle homogenates were resolved on SDS-PAGE, transferred and detected by chemiluminescence with antibodies against phospho-MAPK (panel 1) or phospho-Akt (panel 3). Membranes were stripped and blotted for ERK2 (panel 2) and Akt1 (panel 4) respectively, to normalize for sample loading. A representative blot (from 4-5 separate experiments) for each protein is presented.

FIGS. 7a-7f show glucose and insulin tolerance tests in KO and WT mice. After an overnight fast, an oral glucose load (1 mg/g body weight) (FIGS. 7a, 7b) or intra-peritoneal glucose load (1.5 mg/g body weight) (FIGS. 7c, 7d) was given to 10-week old Ahsg KO and WT mice. Insulin tolerance tests were done on fed (random) mice using an intra-peritoneal injection of 0.75—(FIG. 7e) or 0.15 U regular human insulin/kg body weight (FIG. 7f). Blood glucose (in FIGS. 7a, 7b, 7c, 7e, 7f) or plasma insulin (FIG. 7d) was measured as described in the Examples. Results shown are either from male or female mice (as similar findings were observed in both sexes). Results are expressed as mean±S.E.M.*p<0.05, ** p<0.01, *** p<0.001. WT vs. KO

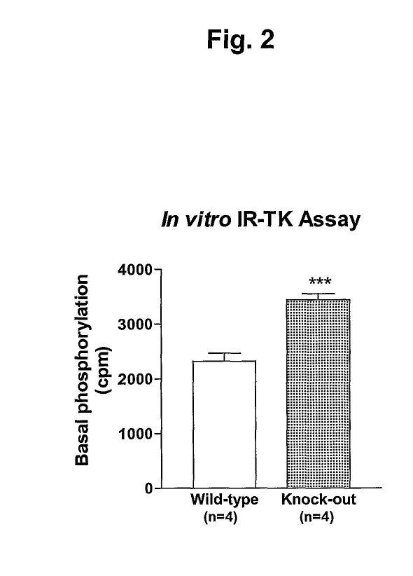

FIGS. 8a-8c show results of euglycemic-hyperinsulinemic clamp studies in conscious KO and WT mice: Glucose infusion rate (FIG. 8a) and 2-DOG uptake in white adipose tissue, soleus and gastrocnemius muscles (FIG. 8b) were determined using the euglycemic-hyperinsulinemic clamp technique in fasted 12-16 week old male mice. Tissue glycogen content (FIG. 8c) was assayed at the end of the euglycemic-hyperinsulinemic clamp. Results are mean±S.E.M. for five animals per genotype. *p<0.05. WT vs. KO

FIGS. 9 and 10 shows results of plasma insulin and homeostasis model assessment (HOMA) in WT and KO mice fed LF or HF diet. After an overnight fast, HF or LF-fed (9 weeks) Ahsg KO and WT mice were given an intraperitoneal glucose tolerance test (1.5 mg glucose/g body weight) and blood glucose and plasma insulin concentrations were measured, *p<0.05, WTHF vs. KOHF (FIG. 9) and HOMA-IR calculated [fasting glucose (mmol/l)×fasting insulin (μU/ml)/22.5], *p<0.05, WTHF vs. WTLF, KOHF or KOLF (FIG. 10). Results are expressed as mean±S.E.M.

FIG. 11 is a schematic diagram of a model of glucose homeostasis involving competition between skeletal muscle and adipose tissue for limiting blood glucose following feeding

### DESCRIPTION OF THE PREFERRED EMBODIMENTS

The principles and foundations of glucose metablism and its disorders, insulin action, insulin dysregulation, insulin resistance, diabetes, and the like can be found in the following texts, the contents of which are hereby incorporated by reference in their entireties: Ellenberg and Rifkin's “Diabetes Mellitus”, 5^{th} edition (or later), Porte and Sherwin (eds)1 Appleton and Lange Press, 1997; Davidson (ed.), Clinical Diabetes Mellitus, 3^{rd} Ed. (or later), Thieme Publications, 2000.

To understand the role of Ahsg in insulin action, the present inventors explored glucose homeostasis in mice carrying two null alleles for Ahsg gene. Since Ahsg was known to inhibit insulin-induced IR-autophosphorylation and TK activity (see Example I), they predicted that genetic ablation of Ahsg would enhance insulin signal transduction and increase whole-body insulin sensitivity. Further, the consequence of this genetic manipulation was examined in an accepted model of acquired insulin resistance, feeding of a high fat (HF) diet, leading to the discovery of a novel obesity-resistance function of Ahsg. Ahsg-null knockout (KO) and wild type (WT) mice were divided into 2 dietary groups within each genotype

(a) high fat diet (40% fat by weight) and

(b) a low fat (LF) group (4% fat by weight).

Both diets used soybean oil as the fat source. See Example II A result obtained after 9 weeks of feeding on these diets ad lib are summarized in the following table: f

Body Weight (grams)

| | Wild Type | Knockout |
|---|---|---|
| High Fat | 28.1 ± 0.9 | 24.4 ± 1 g |
| Low Fat | 24.3 ± 0.9 | 22.5 ± 0.9 |
| P value | <0.005 | N.E. |

Thus, body weight was significantly higher in HF-fed WT mice as compared to LF fed WT mice. However, the body weights of HF-fed KO mice were not significantly different from the LF-fed KO mice. Comparisons of total body fat weight relative to body weight (total fat %) showed the same patterns. Therefore, a HF diet which always induces increased weight gain, does not affect the body weight gain in the absence of Ahsg gene function. Mice lacking genes producing AHSG are resistant to the obesity-producing effect of the HF.

Ahsg-null mice retained their sensitivity to insulin's action of lowering blood glucose. In fact, increased insulin sensitivity was observed in Ahsg-knockout mice. This was confirmed through insulin tolerance tests, insulin signal transduction assays of several signaling molecules, including IR, MAP, Kat, IRS-1 and 2, in liver and/or muscle.

Ahsg-null mice showed markedly enhanced glucose disposal in both oral and intraperitoneal glucose tolerance tests.

These discoveries implicate AHSG as a factor that contributes to obesity. The findings disclosed herein therefore have significant practical implications for treatment of obesity, type 2 diabetes and several other insulin resistant conditions. AHSG can serve as a target protein for therapeutic approaches in the treatment of the above mentioned disease states. According to this invention, inhibitors of AHSG activity, whether they inhibit its phosphorylation, promote its dephosphorylation, inhibit its expression (for example as antisense oligonucleotides or ribozymes), are used to treat a subject to achieve lower body weight and body fat content and/or to improve insulin sensitivity and otherwise counteract the development or progression of Type 2 diabetes, the metabolic syndrome or other disorders associated with insulin resistance.

Until the present invention, evidence of AHSG's role as an inhibitor of insulin-stimulated IR TK had been obtained from studies performed in vitro, in intact cells, isolated tissue and whole animals. The Ahsg KO mouse model enabled characterization of the physiology and molecular basis of insulin action in the absence of AHSG.

Though several other functions of AHSG have been reported in scientific literature, none have been unequivocally established. The present invention points to a critical role for AHSG in the regulation of insulin action, though the physiological function of AHSG is not limited to this.

AHSG Genes and Nucleic Acids

The genomic sequence (and structure) of the gene encoding human AHSG (Osawa, M., Gene 196 (1-2), 121-125 (1997)) is shown below (SEQ ID NO:1) This information is found in GenBank Accession No. D67013. Coding sequences of exons 1-7 are shown in bold.

| | |
|---|---|
| 1 | gatcacagta gaagacattt cctctgctgc caaacccatg |
| | gcactctgag gctgactgtg |
| 61 | tccacctcat tccctcagct gtcttctctt tgctgctatt |
| | accatgttcc aagcagactt |
| 121 | tggagcatct cccccacagc agcatggact ttggcagatt |
| | tcttggggac cagcgatgtc |
| 181 | ctaacctgtt tgcttttcca gggctgatgt ttgcagggtg |
| | tttttttttt tcttttgaac |
| 241 | caaagcagaa atcatcctgt atccttatgc aattcttccg |
| | gcaggctcca acagataaat |
| 301 | aaagcccacc accctccatg ggtctacctt tcccagcaga |
| | gcacctgggt tggtcccgaa |
| 361 | gcctccaacc acctgcacgc ctgcctgcca gggcctctct |
| | ggggcagcca tgaagtccct |
| 421 | cgtcctgctc ctttgtcttg ctcagctctg gggctggcac |
| | tcagccccac atggcccagg |
| 481 | gctgatttat agacaaccga actgcgatga tccagaaact |
| | gaggaagcag ctctggtggc |
| 541 | tatagactac atcaatcaaa accttccttg gggatacaaa |
| | cacaccttga accagattga |
| 601 | tgaagtaaag gtgtggcctc aggtaagtgg acctgctgtc |
| | tatgagctga aataatgtgt |
| 661 | acatggagct caatcaggtg cctcaaaaaa tcaccatcca |
| | cccagtgcaa atgaaaccac |
| 721 | agaggagtaa attctctgat ttcttcccag gagtgaggga |
| | aggggcaggc agagggcagg |
| 781 | agaggagaca ttctgtatgg cagtcatggg tgtcaggagg |
| | gagctgggtg gggtgtgagg |
| 841 | tggtgtgcag gagaaaaagt gcttcaaatg gtagtgtgca |
| | gatcacagac agaaagtgta |
| 901 | acttgctgga aaaactagga cccaagagac cagctcctag |
| | ttgccaagtt accaccggct |
| 961 | gaaaatcacg tatctgtctt tggtttggtt tctctctaac |
| | aaagactgag aatgaataaa |
| 1021 | actagcatct ggcagatgcc tactatatgc caggcccatt |
| | cacatagatt atctcattta |
| 1081 | ctctttcccg gtcctgcctc ctggtgctgt gtggtacata |
| | tattgttctt gtcttaccca |
| 1141 | agaggagacc aaggctctct tgtgtgtgtg tgtgcagttt |
| | tttggttttt ggttttgttt |
| 1201 | tttttttttt ggtccaaaat catataatta ctaagtcttc |
| | aggctgggat ttgattccat |
| 1261 | atctgtgttc caacttctac acaaactgcc tcccaaagag |
| | agttacccac atcccagaga |
| 1321 | gaagtcttgg cataaacaca attcacctcc tcacacacta |
| | gacaggaaac caacgcagct |
| 1381 | tgaagccagt gacaagaaaa atcaagctgg aaatatgcct |
| | cggggatcag tcaagagatt |
| 1441 | tggagaggtg gaaagaagct gtctgcctac tgcctgtttt |
| | gaaattagat ttatttctga |
| 1501 | ttaaggacaa ttctttcagc aaatatgtat tacaagcctt |
| | ccttggacaa gaaccagaga |
| 1561 | tattaggttg aaccatataa aactgccatt tttctatatc |
| | aaaagcaacc aaatattggc |
| 1621 | cgttttaatg gttcaaccta atacagtggt gaaaaaggca |
| | caatatgtgc ccacaagagc |
| 1681 | ttacaatcta ggttggaaaa taaggttcaa caacaggaag |
| | cctggaccga ctgacgactg |
| 1741 | ccatccgtct cacaaagaga caaaatattt gaaatcagga |
| | ttgctccgga tggattttaa |
| 1801 | gagtgctgca gccatattaa agcacagtgg tggttaggag |
| | gaaacgctga tcaagtcagg |
| 1861 | ggaaatgaac acgcaacacg cacatctgag ggaaaaggta |
| | atcatgaatg ggcattgtga |
| 1921 | cttttactaa aggcagagct tcagagttgg ttcccttgag |
| | aaacccaggt gtacccggtt |
| 1981 | cctgttcgcc agagctgtga acgctttcag gcagtcactc |
| | tgggcacacc tggacatcat |
| 2041 | aaaatgcgga acttctccca ggggagggga tgctgaggct |
| | tcaggtacta gtgaatcagg |
| 2101 | cagaaccaat gagaggcaaa cagagctggg ctgagaggag |
| | aaaaggcata cttgtacctt |
| 2161 | ctggtttttc aggttcgaag acaagataca gaaacaggtg |
| | aactcacaag aatatctcca |
| 2221 | aggattgttg caagctccct cgtgtctaca ctagtgacat |
| | ccagtttcct gtcagaggga |
| 2281 | gacatgccct tccccattat cgccagcagg gggaagtaga |
| | gagcagcatc gttgcatgcc |
| 2341 | ggcacctgct gcacaagcca agacaaagga aaaaccaagg |
| | acaacagcag caaaaacctc |
| 2401 | taggagggaa aagaaaacgg aggaaggaag gaaagcaaat |
| | aatgaaaagg aagaaagaaa |
| 2461 | gaaggagagg gagggataga ggagattaaa aggccacagt |
| | aagatattac cctacaccac |
| 2521 | ctattttgca gcttgtctga gaaaaatcca aacttgcatt |
| | ttcccaaagc actgcttgcc |
| 2581 | gagtgaaatc ttaaaaaata aaataaataa taaatacaaa |
| | taagtgttaa cacccatttg |
| 2641 | tagttttcaa atagagcgca gagtgagggc tgtggctcca |
| | tcgacttgtt caagcccagg |
| 2701 | accccgtctg ctttgcgagc atcatctggt gcttccttaa |
| | tcaacagacg aagaccagac |
| 2761 | aagccctggt cattgtcctg cccacaggcc agttcagagc |
| | tagacggagt tgcagactga |
| 2821 | cagtaagaat gacatttccc tcacctctcc aaaagcgggg |
| | tgctctcaag cccaatgagg |
| 2881 | gcgcataccg tggaccgcac cacaggatca ggggaatagg |
| | ttgctcgcgg cttcactctt |
| 2941 | tgtctccaca gcagccctcc ggagagctgt ttgagattga |
| | aatagacacc ccggaaacca |
| 3001 | cctgccatgt gctggacccc acccctgtgg caagatgcag |
| | cgtgaggcag ctgaaggagc |
| 3061 | atgtgagtac ccttcttagg atgactgtag gtggcccttc |
| | ggccagctcc accgattcac |
| 3121 | ccagcgtctc agcctgcctt cttggctagc cagggtgcag |
| | tttctaaaat tgccatttgt |
| 3181 | ggccgagcgc agtggctcat gcctataatc tcagcacttt |
| | gggaggctga ggcaagtgga |
| 3241 | tcgcctgagg tcaggagttc aagaccagcc tggccagtat |
| | ggtgaaaccc catctctact |
| 3301 | aaaaatacaa aaattagctg gacgtggtga cgggcacctg |
| | taaatcccag ctcctcggga |
| 3361 | ggctgaggca ggagaatcgc ttgaacccgg gaggtggagg |
| | ttgcagtgag ccaagatcct |
| 3421 | gccattgcac tccagcctgg gcaacaacag tgaatctcta |
| | tctcgaaata ataataataa |
| 3481 | tcatcatcat cataaataaa attgccattt gatgccactt |
| | gccctggggc tgagttttac |
| 3541 | aagcgtttaa ctatatcgtt gtatccctga aagctgagag |
| | tgccatgttt cagtattacc |
| 3601 | cagcaaaggc gattttgcaa gggtcacctt tgacagccgt |
| | gcctggaggg agcctgcccg |
| 3661 | gggtgcgaag gggaagggca gccatcctca cgtgggtttc |
| | tttctccagg ctgtcgaagg |
| 3721 | agactgtgat ttccagctgt tgaaactaga tggcaagttt |
| | tccgtggtat acgcaaaatg |
| 3781 | tgattccagt ccaggtacag atgactattc ttattctcat |
| | tttttccttg tagagaaagt |
| 3841 | ggggaaggga tctgaataat tttcaactta agtagttcta |
| | gcagctttgt cggtgaggaa |
| 3901 | aaggagaagc caaatttcct gggttctggg atttttaaaa |
| | ttgtgtttta agaagctact |
| 3961 | cttggcctgg tgcggtggct cacgcctgta atccacccac |
| | ccgaggcagg tggatcacct |
| 4021 | gaagtcagaa gttcgagacc agcctggcca acatagtgaa |
| | acccccatct ctactaaaaa |
| 4081 | tacaaaaatg tggtggtgct cgcctgtaat cccagctact |
| | agggaggctg aggcaggaga |
| 4141 | atcgcttgaa cctgggaggc agaggtggca gtgggccgag |
| | atcgcaccac tgcactccag |
| 4201 | cctgagtgac acagagtgag accctgtctc ccaaaaataa |
| | gaagttattc ttactggaag |
| 4261 | tgaaaattgc ctcgtgatga taagagctcc ttcagaaatg |
| | tcagcatagc caaagccttt |
| 4321 | tgaaggttta gtaagaagca gagaaagtgc ctgaagctat |
| | ctggggaatg ccttagccct |
| 4381 | tgctaacgca gcagagctgg ggccatgcca gggagaatgg |
| | ctgcccacat cctggtttcc |
| 4441 | tctctccgag cagactcagc cgaggacgtg cgcaaggtgt |
| | gccaagactg ccccctgctg |
| 4501 | gccccgctga acgacaccag ggtggtgcac gccgcgaaag |
| | ctgccctggc cgccttcaac |
| 4561 | gctcagaaca acggctccaa ttttcagctg gaggaaattt |
| | cccgggctca gcttgtggta |
| 4621 | aagactgaga ttcttttgac aggttgggca gttcggtggc |
| | acttcgggaa tgtactgtac |
| 4681 | gtggtggagc gggaggcagg gcaagaacag gcgcaggggc |
| | agcgatgaga aagcaaggag |
| 4741 | agggttgttt ggaaagggaa gaaagcatcc taagggggta |
| | tgaggctcct gagtgtcatg |
| 4801 | aggaccccaa caccctcagc gcctccccca tgctgagcca |
| | ctgtaacgtc cagcagccac |
| 4861 | agctgccggc aggtacatcc ccactccctc cgttccagct |
| | aaaaccaaag ctcagtgtca |
| 4921 | gctggtagag tttgcccacg tcggccagaa gcactcactg |
| | taaatttgct gggctccagt |
| 4981 | accacccatc tccgctgaac atctgccaca gactcgtaat |
| | taatactcac ttgtgctgac |
| 5041 | aagcttataa tggcaagatc ttaaaatgcc tttcgagtca |
| | ctggagaaaa catctcattg |
| 5101 | tactgtgggt ggtttagcac attggaattc aacagaattc |
| | aaatgtttaa gaaaatgtat |
| 5161 | tctggatatc agccatggcc atacttggaa atacgctagt |
| | atagacggca attctattaa |
| 5221 | tcagaatatg tgattctcag aacatcccca ccccagacta |
| | caccaaataa cagatatttt |
| 5281 | attgtgtcca tatgctccaa ctactttaaa aaagaaaagc |
| | tcaagtgata tcttccatac |
| 5341 | tttcatctaa atcttttcat ttgagcctgc tctatgaaac |
| | aggtggaaga ggtattaatc |
| 5401 | tcttcacttt cccaccctat cttggaataa cctgaacctt |
| | gggtatcaag tgcagcccaa |
| 5461 | gagtgagggc tggggggagg cagggttccc actcctatca |
| | gtctaaggct ggccttctga |
| 5521 | ttccggtttc ctatctggaa actcacctcc accctgaagg |
| | accggtgatg gaaactttcc |
| 5581 | cctcctacaa gggagacaca acccctacct ctaaagcaca |
| | agcacttgag aacacaaccc |
| 5641 | cataacaact tccctatgta aaccattgag ggacatgtct |
| | tctgggccga cgcatggtct |
| 5701 | gcatgaatgg tgctccccga aggaggctac ttcccgctct |
| | ccttctctgc ccltttcatt |
| 5761 | gtaagtcatc tttcctcaag agcattttca tgtactcttc |
| | tcagcccctc ccaccttcta |
| 5821 | cctatgtgga gttcacagtg tgtggcactg actgtgttgc |
| | taaagaggcc acagaggcag |
| 5881 | ccaagtgtaa cctgctggca gaaaaggtga gtgggccggg |
| | accttggggt gttaccactc |
| 5941 | ggacagagct gtttgtggaa cagaacatcc ttggttagtt |
| | tgtttcttgg ggctgcagac |
| 6001 | agagaataac agtgaaaatc ccctctccct gtggatcacg |
| | gaaagcctcc ttttagggtg |
| 6061 | tcacctcatc cctttaagag ctgtcatcaa atcatctcac |
| | ccactggaag cacatgaagt |
| 6121 | taggagaaag agagaggtta tttgttaatg aagccaagtc |
| | acgcccaccc actgggaatg |
| 6181 | tgaagtgcac atttcctaga catataactc tgatacaaaa |
| | gctttcaagt ccttgagcca |
| 6241 | ataatgtaca cttctaggat ttcagtctta agaagtcatc |
| | aagtggccag gcatgatggt |
| 6301 | tcatgcctgt aatccagcac tttgggaggc caagacgggt |
| | ggatcgggag gtcaggagat |
| 6361 | cgagaccatc ctggctaaca tggtgaaacc ccgtctctac |
| | taaaaataca aaaaaattag |
| 6421 | ccaggcttgg tggtgagcgc ctgtagtccc agctactcgg |
| | gaggctgagg caggagaatg |
| 6481 | gtgtgaaccc aggaggcaga tgttgcagta aactaagatc |
| | gtgccactgc actccagcct |
| 6541 | gggcaacaga acgagactct gtctcaagaa aaaaagaaaa |
| | agaaaaagaa ttcctccgtg |
| 6601 | acatttgaca gaatatatct ataaaaatga tttattatgg |
| | atataaagag accaaaaaag |
| 6661 | agagatctgt atgtccaaca ggaaggtgtc attgaataat |
| | ccatgcacat cagtaaatag |
| 6721 | aaaattgtgc agacactaaa aattgtgttt tcaaggaata |
| | atgaatgata tgagaaaatg |
| 6781 | ctattatggc aagtgaaaac acacaggata caacatcgta |
| | tagtcacaat gatctcaatt |
| 6841 | tttaaatcat atttaatagt attttaaaat aagttagaaa |
| | tgcatcaatg ttaacagtcc |
| 6901 | ttctttctag gccaccacca gaaagggatt atgggtaatc |
| | tctctcactc tccaagtatt |
| 6961 | tctgtatttc catgttatat atagaatcat atacctccca |
| | caagcagaaa ctataacttt |
| 7021 | aagaaaaatg gtttttccaa ctaatttaag gttggcgcgt |
| | caatgaaatt gggggggatc |
| 7081 | catttttgaa attagttaaa ataaatcctc tttctctgtg |
| | ggcagcaata tggcttttgt |
| 7141 | aaggcaacac tcagtgagaa gcttggtggg gcagaggttg |
| | cagtgacctg cacggtgttc |
| 7201 | caaacacagg taacagctcc gtgaatattc ttgcctacac |
| | cttcagaata caatgacccc |
| 7261 | ttcacattta tgcagtgcag tagtgatgac aggacatttg |
| | ctctcccgtg cttctgaatc |
| 7321 | tcacagtatg aaataacact ggggtatgcg gaatcatcaa |
| | caaatggaag gatattttag |
| 7381 | ctatgccttt ccctcccacg aactagtgac atacgggaag |
| | aaccatctta ctgtgtagtt |
| 7441 | gacaaagcca cctttttatt tgtgggaggt gggagtggtt |
| | ttctgagttg cagagaccag |
| 7501 | gtggccagat ctacctgtta gctcccagtg gctgcagctt |
| | cagatgacaa agagggtggc |
| 7561 | actgctgggc aagggtgagc cataggtggg gtgcttttac |
| | tcattggaca tatgtgtgta |
| 7621 | agtccaccat cacaaagaca atcctagtga ggccggggca |
| | acataggcca gtcacccctc |
| 7681 | cttgtaacct tgatgacaat cccttgtact taggtaggtc |
| | ctttcttgct agactctttg |
| 7741 | caaataaaaa tgtataatgt gaggaaattg ggtgccagtg |
| | ccacctgggc ctgtgggttg |
| 7801 | tcttgcctgg gaggaggaag caaactaact gaaggaaatg |
| | gtcctttttc cagcccgtga |
| 7861 | cctcacagcc ccaaccagaa ggtgccaatg aagcagtccc |
| | cacccccgtg gtggacccag |
| 7921 | atgcacctcc gtcccctcca cttggcgcac ctggactccc |
| | tccagctggc tcacccccag |
| 7981 | actcccatgt gttactggca gctcctccag gacaccagtt |
| | gcaccgggcg cactacgacc |
| 8041 | tgcgccacac cttcatgggt gtggtctcat tggggtcacc |
| | ctcaggagaa gtgtcgcacc |
| 8101 | cccggaaaac acgcacagtg gtgcagccta gtgttggtgc |
| | tgctgctggg ccagtggttc |
| 8161 | ctccatgtcc ggggaggatc agacacttca aggtctaggc |
| | tagacatggc agagatgagg |
| 8221 | aggtttggca cagaaaacat agccaccatt ttgtccaagc |
| | ctgggcatgg gtggggggcc |
| 8281 | ttgtctgctg gccacgcaag tgtcacatgc gatctacatt |
| | aatatcaagt cttgactccc |
| 8341 | tacttcccgt cattcctcac aggacagaag cagagtgggt |
| | ggtggttatg tttgacagaa |
| 8401 | ggcattaggt tgacaacttg tcatgatttt gacggtaagc |
| | caccatgatt gtgttctctg |
| 8461 | cctctggttg accttacaaa aaccattgga actgtgactt |
| | tgaaaggtgc tcttgctaag |
| 8521 | cttatatgtg cctgttaatg aaagtgcctg aaagaccttc |
| | cttaataaag aaggttctaa |
| 8581 | gctgaatgtg gtcatgctta ttgcgacttc atcccagctc |
| | ccctcacatg catagccttt |
| 8641 | taccccaaca aacacagtgt ccctaatcaa aaccaaagtg |
| | aaaagagaac caaaagagaa |
| 8701 | caaaaacctg ctgtattgcc agatacagga aaaagtgaga |
| | ctaggatc |

Briefly, the exons are at the following nucleotide positions.

| Region | nt positions |
|---|---|
| exon 1 | 362-622* (of which only nt's 410-362 |
| | are coding sdequdnce |
| exon 2 | 2952-3062 |
| exon 3 | 3710-3794 |
| exon 4 | 4454-4617 |
| exon 5 | 5805-5906 |
| exon 6 | 7126-7209 |
| exon 7 | 7854-8584 |

Thus, the coding sequence comprises a rejoined sequence of nt's 410-622, 2952-3062, 3710-3794, 4454-4617, 5805-5906, 7126-7209, and 7854-8198. Regions between these exons are introns and are described below as potential targets for antisense constructs.

Additional features of this gene are: promoter—nt's 1-361; CAAT signal—nt's 269-273; TATA signal—nt's 296-303; 5′UTR—nt's 362-409; and 3′UTR—nt's 8199-8584.

Relevant parts of SEQ ID NO:1 together encode one of at least two known variant or allelic proteins known as form 1 or AHSG*1. The sequence of the protein precursor (SEQ ID NO:2) is:

| AHSG*1 SEQ ID NO: 2 | |
|---|---|
| MKSLVLLLCL AQLWGWHSAP HGPGLIYRQP NCDDPETEEA | 60 |
| ALVAIDYINQ NLPWGYKHTL | |
| NQIDEVKVWP QQPSGELFEI EIDTLETTCH VLDPTPVARC | 120 |
| SVRQLKEHAV EGDCDFQLLK | |
| LDGKFSVVYA KCDSSPDSAE DVRKVCQDCP LLAPLNDTRV | 180 |
| VHAAKAALAA FNAQNNGSNF | |
| QLEEISRAQL VPLPPSTYVE FTVCGTDCVA KEATEAAKCN | 240 |
| LLAEKQYGFC KATLSEKLGG | |
| AEVAVTCTVF QTQPVTSQPQ PEGANEAVPT PVVDPDAPPS | 300 |
| PPLGAPGLPP AGSPPDSHVL | |
| LAAPPGHQLH RAHYDLRHTF MGVVSLGSPS GEVSHPRKTR | 360 |
| TVVQPSVGAA AGPVVPPCPG | |
| RIRHFKV | 367 |

Also shown is a preferred variant with which the present inventors have worked more extensively, known as AHSG*2 (SEQ ID NO:3)

| | |
|---|---|
| MKSLVLLLCL AQLWGCHSAP HGPGLIYRQP NCDDPETEEA | 60 |
| ALVAIDYINQ NLPWGYKHTL | |
| NQIDEVKVWP QQPSGELFEI EIDTLETTCH VLDPTPVARC | 120 |
| SVRQLKEHAV EGDCDFQLLK | |
| LDGKFSVVYA KCDSSPDSAE DVRKVCQDCP LLAPLNDTRV | 180 |
| VHAAKAALAA FNAQNNGSNF | |
| QLEEISRAQL VPLPPSTYVE FTVSGTDCVA KEATEAAKCN | 240 |
| LLAEKQYGFC KATLSEKLGG | |
| AEVAVTCMVF QTQPVSSQPQ PEGANEAVPT PVVDPDAPPS | 360 |
| PPLGAPGLPP AGSPPDSHVL | |
| LAAPPGHQLH RAHYDLRHTF MGVVSLGSPS GEVSHPRKTR | 367 |
| TVVQPSVGAA AGPVVPPCPG | |
| RIRHFKV | |

The AHSG*2 variant (SEQ ID NO:3) is characterized by ATG at position 230 (encoding Met at residue 248) and AGC at position 238 ((encoding Ser at residue 256)). The two substitution variant amino acids are highlighted in by bold/underscore in the ASHG*2 sequence above.

The signal peptide sequence of both proteins above is double underscored, such that the mature secreted protein is a protein of 334 amino acids, residues 34-367 of SEQ ID NO:2 or 3. The first allelic variant (SEQ ID NO:2) is characterized in that it has ACG (encoding Thr) at position 230 in exon 6 (residue 248 in the precursor protein) and ACC (encoding Thr) at position 238 in exon 7 (residue 256 in the precursor protein).

Also shown below is the AHSG*2 which includes a C-terminal fusion to an antigenic epitope (V5 followed by a His tag.)—SEQ ID NO:4. The epitope is shown in bold italic and the His residues are underscored

| | |
|---|---|
| 1 | MKSLVLLLCL AQLWGCHSAP HGPGLIYRQP NCDDPETEEA |
| | ALVAIDYINQ NLPWGYKHTL |
| 61 | NQIDEVKVWP QQPSGELFEI EIDTLETTCH VLDPTPVARC |
| | SVRQLKEHAV EGDCDFQLLK |
| 121 | LDGKFSVVYA KCDSSPDSAE DVRKVCQDCP LLAPLNDTRV |
| | VHAAKAALAA FNAQNNGSNF |
| 181 | QLEEISRAQL VPLPPSTYVE FTVSGTDCVA KEATEAAKCN |
| | LLAEKQYGFC KATLSEKLGG |
| 241 | AEVAVTCMVF QTQPVSSQPQ PEGANEAVPT PVVDPDAPPS |
| | PPLGAPGLPP UAGSPPDSHVL |
| 301 | LAAPPGHQLH RAHYDLRHTF MGVVSLGSPS GEVSHPRKTR |
| | TVVQPSVGAA AGPVVPPCPG |
| 361 | RIRHFKV [symbol] HHHHHH |

Other AHSG sequences relevant to the present invention include the following. Murine AHSG amino acid sequence (SEQ ID NO:5) (including signal peptide), (GenBank Accession #CAA05210) is shown below.

| | |
|---|---|
| MKSLVLLLCF AQLWGCQSAP QGTGLGFREL ACDDPEAEQV | 60 |
| ALLAVDYLNN HLLQGFKQVL | |
| NQIDKVKVWS RQRPFGVVYE MEVDTLETTC HALDPTPLAN | 120 |
| CSVRQLTEHA VEGDCDFHIL | |
| KQDGQFRVMH TQCHSTPDSA EDVRKLCPRC PLLTPFNDTN | 180 |
| VVETVNTALA AFNTQNNGTY | |
| FKLVEISRAQ NVPLPVSTLV EFVIAATDCT AKEVTDPAKC | 240 |
| NLLAEKQHGF CKANLMHNLG | |
| GEEVSVACKL FQTQPQPANA NAVGPVPTAN AALPADPPAS | 300 |
| VVVGPVVVPR GLSDHRTYHD | |
| LRRAFSPVAS VESASGETLH SPKVGQPGAA GPVSPMCPGR | 346 |
| IRHFKI | |

Rat AHSG amino acid sequence (SEQ ID NO:6) (including signal peptide), (GenBank Accession #NM_{—}012898) from Rattus norvegicus is shown below.

| | |
|---|---|
| MKSLVLLLCF AQLWSCQSAP QGAGLGFREL ACDDPRTEHV | 50 |
| ALIAVHYLNK HLLQGFRQIL | |
| NQIDKVKVWS RRPFGQVYEL EIDTLETTCH ALDPTPLANC | 120 |
| SVRQQAEHAV EGDCDFHILK | |
| QDGQFRVLHA QCHSTPDSAE DVRKFCPRCP ILIRFNDTNV | 180 |
| VHTVKTALAA FNAQNNGTYF | |
| KLVEISRAQN VPFPVSTLVE FVIAATDCTG QEVTDPAKCN | 240 |
| LLAEKQYGFC KATLIHRLGG | |
| EEVSVACKLF QTQPQPANAN PAGPAPTVGQ AAPVAPPAGP | 300 |
| PESVVVGPVA VPLGLPDHRT | |
| HHDLRHAFSP VASVESASGE VLHSPKVGQP GDAGAAGPVA | 352 |
| PLCPGRVRYF KI | |

Bovine AHSG amino acid sequence (SEQ ID NO:7) (including signal peptide shown by underscore, italic), (GenBank Accession #X16577) from Bos taurus is shown below.

| | |
|---|---|
| MKSFVLLFCL AQLWGCHSIP LDPVAGYKEP ACDDPDTEQA | 60 |
| ALAAVDYINK HLPRGYKHTL | |
| NQIDSVKVWP RRPTGEVYDI EIDTLETTCH VLDPTPLANC | 120 |
| SVRQQTQHAV EGDCDIHVLK | |
| QDGQFSVLFT KCDSSPDSAE DVRKLCPDCP LLAPLNDSRV | 180 |
| VHAVEVALAT FNAESNGSYL | |
| QLVEISPAQF VPLPVSVSVE FAVAATDCIA KEVVDPTKCN | 240 |
| LLAEKQYGFC KGSVIQKALG | |
| GEDVRVTCTL FQTQPVIPQP QPDGAEAEAP SAVPDAAGPT | 300 |
| PSAAGPPVAS VVVGPSVVAV | |
| PLPLHRAHYD LRHTFSGVAS VESSSGEAFH VGKTPIVGQP | 359 |
| SIPGGPVRLC PGRIRYFKI | |

Therapeutic Approaches to Insulin Resistance and/or Obesity.

Based on the information gleaned from the murine studies described in the Examples, the present invention is directed to methods for treating insulin resistance and/or obesity in a subject by interfering in the function of AHSG. This can be accomplished in a number of ways that are discussed below. One approach is to target an antisense nucleic acid to a sequence of the Ahsg gene or mRNA to block ultimately expression of that gene and result in a subject who is effectively similar to a KO mouse as described herein.

Antisense Nucleic Acids

Gene expression involves the transcription of pre-messenger RNA (pre-mRNA) from a DNA template, the processing of the pre-mRNA into mature mRNA, and the translation of the mRNA into one or more polypeptides. The use of antisense DNA or RNA to inhibit RNA function within cells and whole organism has generated much recent interest. Antisense RNA can bind in a highly specific manner to its complementary sequences (“sense DNA or RNA”). This blocks the processing and translation of the sense RNA and may even disrupt interactions with sequence-specific RNA binding proteins. For example, a plasmid was constructed having a promoter which directed the transcription of a RNA complementary to the normal thymidine kinase (TK) mRNA. When such plasmids, together with plasmids containing a normally expressed TK gene, were injected into mutant murine L cells lacking TK, the presence of the antisense gene substantially reduced expression of TK from the normal plasmid (Izant et al., 1984 Cell 36:1007).

Antisense oligonucleotides are inhibitory in various viral systems. For example, Rous sarcoma virus (RSV; a retrovirus) (Zamecnik et al., 1978 Biochemistry 75:280-284) was inhibited by addition to the culture medium of an oligodeoxynucleotide complementary to 13 nucleotides of the 3′ and 5′ LTRs. The DNA was terminally blocked to reduce its susceptibility to exonucleases. It was speculated that this antisense DNA might act by blocking circularization, DNA integration, DNA transcription, translation initiation or ribosomal association. Chang et al., J. Virol. 61:921-24 (1987) inhibited RSV using antisense RNA hybridized to the coding region or to the 5′ or 3′ flanking regions of the viral env gene. Gupta, J. Biol. Chem. 262:7492-96 (1987) inhibited translation of the Sendai virus nucleocapsid protein (NP) and phosphoprotein (P.C) mRNAs by means of antisense DNAs complementary to the 5′ flanking region.

The constitutive expression of antisense RNA in cells has been shown to inhibit the expression of about 20 different genes in mammals and plants, and the list continually grows (Hambor, J. E. et al., J. Exp. Med. 168:1237-1245 (1988); Holt, J. T. et al., Proc. Nat. Acad. Sci. 83:4794-4798 (1986); Izant et al., supra; Izant, J. G. et al., Science 229:345-352 (1985) and De Benedetti, A. et al., Proc. Nat. Acad. Sci. 84:658-662 (1987)). Possible mechanisms for the antisense effect are the blockage of translation or prevention of splicing, both of which have been observed in vitro. Interference with splicing allows the use of intron sequences (Munroe, S. H., EMBO. J. 7:2523-2532 (1988) which should be less conserved and therefore result in greater specificity in inhibiting expression of, ee.g., an enzyme of one species

The antisense oligonucleotides or polynucleotide of the present invention may range from 6 to 50 nucleotides, and may be as large as 100 or 200 nucleotides. Preferred lengths are in the range of 16-30 nucleotides. For the sake of convenience they are referred to herein as “oligonucleotides” even if longer than that which is usually considered to be “oligo.” The oligonucleotides can be DNA or RNA or chimeric mixtures or derivatives or modified versions thereof, single-stranded or double-stranded.

The oligonucleotides can be modified at the base moiety, sugar moiety, or phosphate backbone. The oligonucleotide may include other appending groups such as peptides, or agents facilitating transport across the cell membrane (see, e.g. Letsinger et al., 1989, Proc. Natl. Acad. Sci. U.S.A. 84:684-652; PCT Publication No. WO 88/09810, published Dec. 15, 1988) or blood-brain barrier (see, e.g. PCT Publication No. WO 89/10134, published Apr. 25, 1988), hybridization-triggered cleavage agents (see, e.g. Krol et al., 1988, BioTechniques 6:958-976) or intercalating agents (see, e.g. Zon, 1988, Pharm. Res 5:539-549).

The present invention provides antisense oligonucleotides complementary to a part of the Ahsg gene or an mRNA encoded thereby which can be used therapeutically or in screening methods to identify agents capable of stimulating or inhibiting AHSG induction or action.

Such antisense oligonucleotides are antisense to DNA or RNA encoding AHSG or a portion thereof, or to flanking sequences in genomic DNA which are involved in regulating AHSG gene expression. Introns are known to be useful target sequences. The intronic sequences are shown above (see SEQ ID NO:1, non-bolded nucleotides between exons).

“Antisense” as used herein refers to a nucleic acid having some sequence complementarity such that an antisense DNA or RNA molecule can hybridize with a target mRNA such that translation of the mRNA is inhibited, irrespective of the precise mechanism of inhibition. The antisense nucleic acid of the present invention may be complementary to, or hybridizable to, any one of several portions of the target AHSG DNA or RNA. The action of the antisense nucleotide results in specific inhibition of AHSG gene expression in cells. See: Albers, B. et al., MOLECULAR BIOLOGY OF THE CELL, 2nd Ed., Garland Publishing, Inc., New York, N.Y. (1989), in particular, pages 195-196, which reference is hereby incorporated by reference).

The antisense oligonucleotide may be complementary to any portion of the AHSG sequence. In one preferred embodiment, the antisense oligonucleotide has between about 6 and 30 nucleotides, and is complementary to the initiation ATG codon and an upstream, non-coding translation initiation site of the AHSG sequence. Such antisense nucleotides specific largely for non-coding sequence, are known to be effective inhibitors of the expression of genes encoding other transcription factors (Branch, M. A. 1993 Molec. Cell. Biol. 13:4284-4290).

In another embodiment, the antisense oligonucleotide is selected to be complementary to a portion of the AHSG mRNA sequence encoding a portion of AHSG protein that is most dissimilar from other proteins. Because this part of the AHSG sequence has less homology to other proteins, e.g., family members, etc., such an antisense construct would allow selective more inhibition of AHSG while having less effect on expression of other members of the same family of proteins.

Preferred antisense oligonucleotides are complementary to a portion of the mRNA encoding AHSG, including one or more of exon 1, exon 2, exon 3, exon 4, exon 5, exon 6 or exon 7 of SEQ ID NO:1.

As is readily discernible by one of ordinary skill in the art, the minimal amount of sequence homology required by the present invention is that sufficient to result in sufficient complementarity to provide recognition of the specific target DNA or RNA and inhibition of its transcription, translocation, translation or function while not affecting function of other mRNA molecules and the expression of other genes.

While the antisense oligonucleotides of the invention comprise sequences complementary to at least a portion of an RNA transcript AHSG, absolute complementarity, although preferred, is not required. A sequence “complementary to at least a portion of an RNA,” as referred to herein, means a sequence having sufficient complementarily to be able to hybridize with the RNA, forming a stable duplex; in the case of double-stranded antisense nucleic acids, a single strand of the duplex DNA may thus be tested, or triplex formation may be assayed. The ability to hybridize will depend on both the degree of complementarity and the length of the antisense nucleic acid. Generally, the longer the hybridizing nucleic acid, the more base mismatches with the AHSG target sequence it may contain and still form a stable duplex (or triplex, as the case may be). One skilled in the art can ascertain a tolerable degree of mismatch by use of standard procedures to determine the melting point of the hybridized complex.

The antisense oligonucleotide of the invention can be double-stranded or single-stranded RNA or DNA or a modification or derivative thereof, which can be directly administered to a cell, or which can be produced intracellularly by transcription of exogenously introduced nucleic acid sequences. Thus, antisense RNA may be delivered to a cell by transformation, transfection or infection with a vector into which has been placed DNA encoding the antisense RNA with the appropriate regulatory sequences, including a promoter, to result in expression of the antisense RNA in a host cell.

An oligonucleotide, between about 6 and about 100 bases in length and complementary to the target sequence of AHSG may be synthesized chemically from natural mononucleosides or, alternatively, from mononucleosides having substitutions at the non-bridging phosphorous bound oxygens. Alternatively, the oligonucleotide may be produced by recombinant means.

A preferred mononucleoside analogue is a methylphosphonate analogue of the naturally occurring mononucleosides. More generally, the mononucleoside analogue is any analogue whose use results in an oligonucleotide which has improved diffusion through cell membranes or increased resistance to nuclease digestion within the body of a subject (Miller, P. S. et al., Biochemistry 20:1874-1880 (1981)). Such nucleoside analogues are well-known in the art, and their use in the inhibition of gene expression has been disclosed. See, for example, Miller, P. S. et al., supra.

The antisense oligonucleotide molecule of the present invention may be a native DNA or RNA molecule or an analogue of DNA or RNA. The present invention is not limited to use of any particular DNA or RNA analogue, provided it is capable of adequate hybridization to the complementary genomic DNA (or mRNA) of AHSG, has adequate resistance to nucleases, and adequate bioavailability and cell uptake. DNA or RNA may be made more resistant to in vivo degradation by enzymes such as nucleases, by modifying internucleoside linkages (e.g., methylphosphonates or phosphorothioates) or by incorporating modified nucleosides (e.g., 2′-0-methylribose or 1′-α-anomers).

The naturally occurring linkage is

3′O
O—P═O
O 5′

Alternative linkages include the following:

| | |
|---|---|
| 3′O | 3′O |
| S⁻—P═O | CH₃—P═O |
| O5′ | O5′ |
| 3′O | 3′O |
| NR₂—P═O | RO—P═O |
| O5′ | O5′ |

(where R is H and/or alkyl)

or

3′O
S⁻—P═S
O5′

It is also possible to replace the 3′O—P—O(5′) with other linkages such as (3′)O—CH₂C(O)O(5′), (3′)O—C(O)—NH(5′), and (3′)C—CH₂CH₂S—C(5′).

The antisense oligonucleotide may comprise at least one modified base moiety which is selected from the group including but not limited to 5-fluorouracil, 5-bromouracil, 5-chlorouracil, 5-iodouracil, hypoxanthine, xanthine, 4-acetylcytosine, 5-(carboxyhydroxylmethyl)uracil, 5-carboxymethylaminomethyl-ω-thiouridine, 5-carboxymethylaminomethyl uracil, dihydrouracil, β-D-galactosylqueosine, inosine, N6-isopentenyladenine, 1-methylguanine, 3-methyl-cytosine, 5-methylcytosine, N6-adenine, 7-methylguanine, 5-methylaminomethyluracil, 5-methoxyamino-methyl-2-thiouracil, β-D-mannosylqueosine, 5-methoxy-carboxymethyluracil, 5-methoxyuracil-2-methylthio-N6-isopentenyladenine, uracil-5-oxyacetic acid, butoxosine, pseudouracil, queosine, 2-thio-cytosine, 5-methyl-2-thiouracil, 2-thiouracil, 4-thiouracil, 5-methyluracil, uracil-5-oxyacetic acid methylester, uracil-t-oxyacetic acid, 5-methyl-2-thiouracil, 3(3-amino-3-N-2-carboxypropyl)uracil and 2,6-diaminopurine.

In another embodiment, the oligonucleotide comprises at least one modified sugar moiety selected from the group including, but not limited to arabinose, 2-fluoroarabinose, xylulose, and hexose.

In yet another embodiment, the oligonucleotide comprises at least one modified phosphate backbone selected from the group consisting of a phosphorothioate, a phosphoridothioate, a phosphoramidothioate, a phosphoramidate, a phosphordiimidate, a methylsphosphonate, an alkyl phosphotriester, and a formacetal or analog thereof.

In yet another embodiment, the oligonucleotide is an α-anomeric oligonucleotide which forms specific double-stranded hybrids with complementary RNA in which, contrary to the usual β-units, the strands run parallel to each other (Gautier et al., 1987, Nucl. Acids Res. 15:6625-6641).

In oligonucleotide may be conjugated to another molecule, e.g., a peptide, a hybridization triggered cross-linking agent, a transport agent, a hybridization-triggered cleavage agent, etc., all of which are well-known in the art.

Oligonucleotides of this invention may be synthesized by standard methods known in the art, e.g. by use of an automated DNA synthesizer (such as are commercially available from Biosearch, Applied Biosystems, etc.). As examples, phosphorothioate oligonucleotides may be synthesized by the method of Stein et al., 1988 Nucl. Acids Res. 16:3209, methylphosphonate oligonucleotides can be prepared by use of controlled pore glass polymer supports (Sarin et al., 1988 Proc. Natl. Acad. Sci. U.S.A. 85:7448-7451), etc.

Basic procedures for constructing recombinant DNA and RNA molecules in accordance with the present invention are disclosed by Sambrook, J. et al., In: Molecular Cloning: A Laboratory Manual, Second Edition, Cold Spring Harbor Press, Cold Spring Harbor, N.Y. (1989), which reference is herein incorporated by reference.

Oligonucleotide molecules having a strand which encodes antisense RNA complementary to an AHSG sequence can be prepared using procedures which are well known to those of ordinary skill in the art (Belagaje, R., et al., J. Biol. Chem. 254:5765-5780 (1979); Maniatis, T., et al., In: MOLECULAR MECHANISMS IN THE CONTROL OF GENE EXPRESSION, Nierlich, D. P., et al., eds., Acad. Press, N.Y. (1976); Wu, R., et al., Prog. Nucl. Acid Res. Molec. Biol. 21:101-141 (1978); Khorana, H. G., Science 203:614-625 (1979)). Automated synthesizers may be used for DNA synthesis. Techniques of nucleic acid hybridization are disclosed by Sambrook et al (supra), and by Haymes, B. D., et al., In: NUCLEIC ACID HYBRIDIZATION, A PRACTICAL APPROACH, IRL Press, Washington, DC (1985)), which references are herein incorporated by reference.

Thus, the antisense nucleic acid of the invention may be produced intracellularly by transcription from an exogenous sequence. For example, a vector can be introduced in vivo such that it is taken up by a cell, within which cell the vector or a portion thereof is transcribed, producing an antisense nucleic acid (RNA) of the invention. Such a vector can remain episomal or become chromosomally integrated, as long as it can be transcribed to produce the desired antisense RNA. Vectors, which are discussed in more detail below, can be constructed by recombinant DNA technology methods standard in the art. Vectors can be plasmid, viral, or others know in the art, used for replication and expression in mammalian cells. Expression of the sequence encoding the antisense RNA can be by any promoter known in the art to act in mammalian, preferably human, cells (see below).

Nucleic Acids

As noted, the antisense nucleic acid molecule preferably comprises a nucleotide sequence that hybridizes with SEQ ID NO:1 or with a rearranged product thereof that encodes AHSG, or with AHSG mRNA, or with any nucleic acid that encodes a protein of human origin having the sequence SEQ ID NO:2 or 3, or SEQ ID NO:5. (of murine origin), or SEQ ID NO:6 (rat origin) or SEQ ID NO:7 (bovine origin). The invention is also directed to an isolated nucleic acid that hybridizes with the above nucleic acid molecule under stringent hybridization conditions. Preferred stringent conditions include incubation in 6× sodium chloride/sodium citrate (SSC) at about 45° C., followed by a wash in about 0.2×SSC at a temperature of about 50° C. A preferred nucleic acid molecule is antisense to a nucleic acid molecule that encodes (a) a protein having an amino acid sequence selected from SEQ ID NO:2 and SEQ ID NO:3 or (b) a biologically active fragment, homologue or other functional derivative of the protein.

In reference to a nucleotide sequence, the term “equivalent” is intended to include sequences encoding structurally homologous and/or a functionally equivalent proteins. For example, a natural polymorphism of AHSG nucleotide sequence (especially at the third base of a codon) may be manifest as “silent” mutations which do not change the amino acid sequence. However, polymorphisms that involve amino acid sequence changes in AHSG, do exist (see above, and others may exist in a human (or other mammalian) population. Those of skill in the art will appreciate that these allelic variants that have changes in one or more nucleotides (up to about 3-4% of the total coding sequence) will likely be found in a human population due to natural allelic variation. Antisense oligo-or polynucleotides that have the sequence corresponding to any and all such allelic variations that result in nucleic acid polymorphisms in the DNA encoding AHSG are within the scope of the invention.

Furthermore, there may be one or more naturally occurring isoforms or related, immunologically cross-reactive family members of the AHSG protein described herein that is the target of the antisense approach described herein. Such isoforms or family members are defined as proteins that share function amino acid sequence similarity to AHSG, even if they are encoded by genes at different loci.

Nucleic acid sequences of this invention may also include linker sequences, natural or modified restriction endonuclease sites and other sequences that are useful for manipulations related to cloning, antisense based inhibition, or, in the case of an AHSG nucleic acid, expression or purification of encoded protein or fragment thereof. These and other modifications of nucleic acid sequences are described herein or are well-known in the art.

Vector Construction

Construction of suitable vectors containing the desired coding and control sequences employs standard ligation and restriction techniques which are well understood in the art. Isolated plasmids, DNA sequences, or synthesized oligonucleotides are cleaved, tailored, and re-ligated in the form desired.

The DNA sequences which form the vectors are available from a number of sources. Backbone vectors and control systems are generally found on available “host” vectors which are used for the bulk of the sequences in construction. For the pertinent coding sequence, initial construction may be, and usually is, a matter of retrieving the appropriate sequences from cDNA or genomic DNA libraries. However, once the sequence is disclosed it is possible to synthesize the entire gene sequence in vitro starting from the individual nucleotide derivatives. The entire gene sequence for genes of sizable length, e.g., 500-1000 bp may be prepared by synthesizing individual overlapping complementary oligonucleotides and filling in single stranded nonoverlapping portions using DNA polymerase in the presence of the deoxyribonucleotide triphosphates. This approach has been used successfully in the construction of several genes of known sequence. See, for example, Edge, M. D., Nature (1981) 292:756; Nambair, K. P., et al., Science (1984) 223:1299; and Jay, E., J Biol Chem (1984) 259:6311.

Synthetic oligonucleotides are prepared by either the phosphotriester method as described by references cited above or the phosphoramidite method as described by Beaucage, S. L., and Caruthers, M. H., Tet Lett (1981) 22:1859; and Matteucci, M. D., and Caruthers, M. H., J Am Chem Soc (1981) 103:3185 and can be prepared using commercially available automated oligonucleotide synthesizers. Kinase treatment of single strands prior to annealing or for labeling is achieved using an excess, e.g., about 10 units of polynucleotide kinase to 1 nmole substrate in the presence of 50 mM Tris, pH 7.6, 10 mM MgCl₂, 5 mM dithiothreitol, 1-2 mM ATP, 1.7 pmoles γ-³²P-ATP (2.9 mCi/mmole), 0.1 mM spermidine, 0.1 mM EDTA.

Once the components of the desired vectors are thus available, they can be excised and ligated using standard restriction and ligation procedures. Site-specific DNA cleavage is performed by treating with the suitable restriction enzyme (or enzymes) under conditions which are generally understood in the art, and the particulars of which are specified by the manufacturer of these commercially available restriction enzymes. See, e.g., New England Biolabs, Product Catalog. In general, about 1 mg of plasmid or DNA sequence is cleaved by one unit of enzyme in about 20 ml of buffer solution; in the examples herein, typically, an excess of restriction enzyme is used to insure complete digestion of the DNA substrate. Incubation times of about one hour to two hours at about 37° C. are workable, although variations can be tolerated. After each incubation, protein is removed by extraction with phenol/chloroform, and may be followed by ether extraction, and the nucleic acid recovered from aqueous fractions by precipitation with ethanol. If desired, size separation of the cleaved fragments may be performed by polyacrylamide gel or agarose gel electrophoresis using standard techniques. A general description of size separations is found in Methods in Enzymology (1980) 65:499-560.

Restriction cleaved fragments may be blunt ended by treating with the large fragment of E. coli DNA polymerase I (Klenow) in the presence of the four deoxynucleotide triphosphates (dNTPs) using incubation times of about 15 to 25 min at 20° to 25° C. in 50 mM Tris pH 7.6, 50 mM NaCl, 6 mM MgCl₂, 6 mM DTT and 0.1-1.0 mM dNTPs. The Klenow fragment fills in at 5′ single-stranded overhangs but chews back protruding 3′ single strands, even though the four dNTPs are present. If desired, selective repair can be performed by supplying only one of the, or selected, dNTPs within the limitations dictated by the nature of the overhang. After treatment with Klenow, the mixture is extracted with phenol/chloroform and ethanol precipitated. Treatment under appropriate conditions with S1 nuclease or BAL-3 1 results in hydrolysis of any single-stranded portion.

Ligations are typically performed in 15-50 ml volumes under the following standard conditions and temperatures: for example, 20 mM Tris-HCl pH 7.5, 10 mM MgCl₂, 10 mM DTT, 33 μg/ml BSA, 10-50 mM NaCl, and either 40 μM ATP, 0.01-0.02 (Weiss) units T4 DNA ligase at 0° C. (for “sticky end” ligation) or 1 mM ATP, 0.3-0.6 (Weiss) units T4 DNA ligase at 14° C. (for “blunt end” ligation). Intermolecular “sticky end” ligations are usually performed at 33-100 μg/ml total DNA concentrations (5-100 nM total end concentration). Intermolecular blunt end ligations are performed at 1 mM total ends concentration.

In vector construction employing “vector fragments”, the fragment is commonly treated with bacterial alkaline phosphatase (BAP) or calf intestinal alkaline phosphatase (CIAP) in order to remove the 5′ phosphate and prevent self-ligation. Digestions are conducted at pH 8 in approximately 10 mM Tris-HCl, 1 mM EDTA using BAP or CIAP at about 1 unit/mg vector at 60° for about one hour. The preparation is extracted with phenol/chloroform and ethanol precipitated. Alternatively, re-ligation can be prevented in vectors which have been double digested by additional restriction enzyme and separation of the unwanted fragments.

Any of a number of methods are used to introduce mutations into the coding sequence to generate variants of the invention. These mutations include simple deletions or insertions, systematic deletions, insertions or substitutions of clusters of bases or substitutions of single bases.

For example, modifications are created by site-directed mutagenesis, a well-known technique for which protocols and reagents are commercially available (Zoller, M J et al., Nucleic Acids Res (1982) 10:6487-6500 and Adelman, J P et al., DNA (1983) 2:183-193)). Correct ligations for plasmid construction are confirmed, for example, by first transforming E. coli strain MC1061 (Casadaban, M., et al., J Mol Biol (1980) 138:179-207) or other suitable host with the ligation mixture. Using conventional methods, transformants are selected based on the presence of the ampicillin-, tetracycline- or other antibiotic resistance gene (or other selectable marker) depending on the mode of plasmid construction. Plasmids are then prepared from the transformants with optional chloramphenicol amplification optionally following chloramphenicol amplification ((Clewell, D B et al., Proc Natl Acad Sci USA (1969) 62:1159; Clewell, D. B., J Bacteriol (1972) 110:667). Several mini DNA preps are commonly used. See, e.g., Holmes, D S, et al., Anal Biochem (1981) 114:193-197; Birnboim, H C et al., Nucleic Acids Res (1979) 7:1513-1523. The isolated DNA is analyzed by restriction and/or sequenced by the dideoxy nucleotide method of Sanger (Proc Natl Acad Sci USA (1977) 74:5463) as farther described by Messing, et al., Nucleic Acids Res (1981) 9:309, or by the method of Maxam et al. Methods in Enzymology (1980) 65:499.

Vector DNA can be introduced into mammalian cells via conventional techniques such as calcium phosphate or calcium chloride co-precipitation, DEAE-dextran-mediated transfection, lipofection, or electroporation. Suitable methods for transforming host cells can be found in Sambrook et al. supra and other standard texts and are discussed in more detail below.

Inducible expression vectors include pTrc (Amann et al., (1988) Gene 69: 301-315) and pET 11d (Studier et al., Gene Expression Technology: Methods in Enzymology 185, Academic Press, San Diego, Calif. (1990) 60-89). While target gene expression relies on host RNA polymerase transcription from the hybrid trp-lac fusion promoter in pTrc, expression of target genes inserted into pET 11d relies on transcription from the T7 gn10-lacO fusion promoter mediated by coexpressed viral RNA polymerase (T7gn1). Th is viral polymerase is supplied by host strains BL21(DE3) or HMS174(DE3) from a resident λ prophage harboring a T7gn1 under the transcriptional control of the lacUV 5 promoter.

Promoters and Enhancers

A promoter region of a DNA or RNA molecule binds RNA polymerase and promotes the transcription of an “operably linked” nucleic acid sequence. As used herein, a “promoter sequence” is the nucleotide sequence of the promoter which is found on that strand of the DNA or RNA which is transcribed by the RNA polymerase. Two sequences of a nucleic acid molecule, such as a promoter and a coding sequence, are “operably linked” when they are linked to each other in a manner which permits both sequences to be transcribed onto the same RNA transcript or permits an RNA transcript begun in one sequence to be extended into the second sequence. Thus, two sequences, such as a promoter sequence and a coding sequence of DNA or RNA are operably linked if transcription commencing in the promoter sequence will produce an RNA transcript of the operably linked coding sequence. In order to be “operably linked” it is not necessary that two sequences be immediately adjacent to one another in the linear sequence.

The preferred promoter sequences of the present invention must be operable in mammalian cells and may be either eukaryotic or viral promoters. Useful promoters and regulatory elements are discussed below. Suitable promoters may be inducible, repressible or constitutive. An example of a constitutive promoter is the viral promoter MSV-LTR, which is efficient and active in a variety of cell types, and, in contrast to most other promoters, has the same enhancing activity in arrested and growing cells. Other preferred viral promoters include that present in the CMV-LTR (from cytomegalovirus) (Bashart, M. et al., Cell 41:521 (1985)) or in the RSV-LTR (from Rous sarcoma virus) (Gorman, C. M., Proc. Natl. Acad. Sci. USA 79:6777 (1982). Also useful are the promoter of the mouse metallothionein I gene (Hamer, D., et al., J. Mol. Appl. Gen. 1:273-288 (1982)); the TK promoter of Herpes virus (McKnight, S., Cell 31:355-365 (1982)); the SV40 early promoter (Benoist, C., et al., Nature 290:304-310 (1981)); and the yeast gal4 gene promoter (Johnston, S. A., et al., Proc. Natl. Acad. Sci. (USA) 79:6971-6975 (1982); Silver, P. A., et al., Proc. Natl. Acad. Sci. (USA) 81:5951-5955 (1984)). Other illustrative descriptions of transcriptional factor association with promoter regions and the separate activation and DNA binding of transcription factors include: Keegan et al., Nature (1986) 231:699; Fields et al., Nature (1989) 340:245; Jones, Cell (1990) 61:9; Lewin, Cell (1990) 61:1161; Ptashne et al., Nature (1990) 346:329; Adams et al., Cell (1993) 72:306. The relevant disclosure of all of these above-listed references is hereby incorporated by reference.

The promoter region may further include an octamer region which may also function as a tissue specific enhancer, by interacting with certain proteins found in the specific tissue. The enhancer domain of the DNA construct of the present invention is one which is specific for the target cells to be transfected, or is highly activated by cellular factors of such target cells. Examples of vectors (plasmid or retrovirus) are disclosed in (Roy-Burman et al., U.S. Pat. No. 5,112,767). For a general discussion of enhancers and their actions in transcription, see, Lewin, B. M., Genes IV, Oxford University Press, Oxford, (1990), pp. 552-576. Particularly useful are retroviral enhancers (e.g., viral LTR). The enhancer is preferably placed upstream from the promoter with which it interacts to stimulate gene expression. For use with retroviral vectors, the endogenous viral LTR may be rendered enhancer-less and substituted with other desired enhancer sequences which confer tissue specificity or other desirable properties such as transcriptional efficiency.

The nucleic acid sequences of the invention can also be chemically synthesized using standard techniques. Various methods of chemically synthesizing polydeoxynucleotides are known, including solid-phase synthesis which, like peptide synthesis, has been fully automated with commercially available DNA synthesizers (See, e.g., Itakura et al. U.S. Pat. No. 4,598,049; Caruthers et al. U.S. Pat. No. 4,458,066; and Itakura U.S. Pat. Nos. 4,401,796 and 4,373,071, incorporated by reference herein).

Nucleic Delivery to Cells and Animals

DNA delivery, for example to effect what is generally known as “gene therapy” involves introduction of a “foreign” DNA into a cell and ultimately, into a live animal. Several general strategies have been studied and have been reviewed extensively (Yang, N-S., Crit. Rev. Biotechnol. 12:335-356 (1992); Anderson, W. F., Science 256:808-813 (1992); Miller, A. S., Nature 357:455-460 (1992); Crystal, R. G., Amer. J. Med. 92(suppl 6A):44S-52S (1992); Zwiebel, J. A. et al., Ann. N.Y. Acad. Sci. 618:394-404 (1991); McLachlin, J. R. et al., Prog. Nucl. Acid Res. Molec. Biol. 38:91-135 (1990); Kohn, D. B. et al., Cancer Invest. 7:179-192 (1989), which references are herein incorporated by reference in their entirety).

One approach comprises nucleic acid transfer into primary cells in culture followed by autologous transplantation of the ex vivo transformed cells into the host, either systemically or into a particular organ or tissue.

For accomplishing the objectives of the present invention, nucleic acid therapy would be accomplished by direct transfer or transfection of a the functionally active DNA into mammalian somatic tissue or organ in vivo. Transfection is the general process of bringing foreign DNA into cells and obtaining and monitoring protein expression. Common transfection techniques include calcium phosphate coprecipitation, electroporation, and the use of viral vectors, each with its advantages and disadvantages (see below). Cationic liposome-mediated transfection methods (lipofection, cytofection) were an important addition to the previous methods. Additional classes of compounds known to mediate transfection include lipopolyamines and dendrimers.

DNA transfer can be achieved using a number of approaches described below. These systems can be tested for successful expression in vitro by use of a selectable marker (e.g., G418 resistance) to select transfected clones expressing the DNA, followed by detection of the presence of the AHSG expression product (after treatment with the inducer in the case of an inducible system) using an antibody to the product in an appropriate immunoassay. Efficiency of the procedure, including DNA uptake, plasmid integration and stability of integrated plasmids, can be improved by linearizing the plasmid DNA using known methods, and co-transfection using high molecular weight mammalian DNA as a “carrier”.

Examples of successful “gene transfer” reported in the art include: (a) direct injection of plasmid DNA into mouse muscle tissues, which led to expression of marker genes for an indefinite period of time (Wolff, J. A. et al., Science 247:1465 (1990); Acsadi, G. et al., The New Biologist 3:71 (1991)); (b) retroviral vectors are effective for in vivo and in situ infection of blood vessel tissues; (c) portal vein injection and direct injection of retrovirus preparations into liver effected gene transfer and expression in vivo (Horzaglou, M. et al., J. Biol. Chem. 265:17285 (1990); Koleko, M. et al., Human Gene Therapy 2:27 (1991); Ferry, N. et al., Proc. Natl. Acad. Sci. USA 88:8387 (1991)); (d) intratracheal infusion of recombinant adenovirus into lung tissues was effective for in vivo transfer and prolonged expression of foreign genes in lung respiratory epithelium (Rosenfeld, M. A. et al., Science 252:431 (1991); (e) Herpes simplex virus vectors achieved in vivo gene transfer into brain tissue (Ahmad, F. et al., eds, Miami Short Reports—Advances in Gene Technology: The Molecular Biology of Human Genetic Disease, Vol 1, Boerringer Mannheim Biochemicals, USA, 1991).

Retroviral-mediated human therapy utilizes amphotrophic, replication-deficient retrovirus systems (Temin, H. M., Human Gene Therapy 1:111 (1990); Temin et al., U.S. Pat. No. 4,980,289; Temin et al., U.S. Pat. No. 4,650,764; Temin et al., U.S. Pat. No. 5,124,263; Wills, J. W. U.S. Pat. No. 5,175,099; Miller, A. D., U.S. Pat. No. 4,861,719). Such vectors have been used to introduce functional DNA into human cells or tissues, for example, the adenosine deaminase gene into lymphocytes, the NPT-II gene and the gene for tumor necrosis factor into tumor infiltrating lymphocytes. Retrovirus-mediated gene delivery generally requires target cell proliferation for gene transfer (Miller, D. G. et al., Mol. Cell. Biol. 10:4239 (1990). This condition is met by certain of the preferred target cells into which the present DNA molecules are to be introduced, i.e., actively growing tumor cells. Gene therapy of cystic fibrosis using transfection by plasmids using any of a number of methods and by retroviral vectors has been described by Collins et al., U.S. Pat. No. 5,240,846.

The DNA molecules encoding the AHSG sequences may be packaged into retrovirus vectors using packaging cell lines that produce replication-defective retroviruses, as is well-known in the art (see, for example, Cone, R. D. et al., Proc. Natl. Acad. Sci. USA 81:6349-6353 (1984); Mann, R. F. et al., Cell 33:153-159 (1983); Miller, A. D. et al., Molec. Cell. Biol. 5:431-437 (1985),; Sorge, J., et al., Molec. Cell. Biol. 4:1730-1737 (1984); Hock, R. A. et al., Nature 320:257 (1986); Miller, A. D. et al., Molec. Cell. Biol. 6:2895-2902 (1986). Newer packaging cell lines which are efficient an safe for gene transfer have also been described (Bank et al., U.S. Pat. No. 5,278,056.

This approach can be utilized in a site specific manner to deliver the retroviral vector to the tissue or organ of choice. Thus, for example, a catheter delivery system can be used (Nabel, E G et al., Science 244:1342 (1989)). Such methods, using either a retroviral vector or a liposome vector, are particularly useful to deliver the nucleic acid to be expressed to a blood vessel wall, or into the blood circulation of a particular tissue or organ. For AHSG inhibition, liver delivery is expected to be most effective.

Other virus vectors may also be used, including recombinant adenoviruses (Horowitz, M. S., In: Virology, Fields, B N et al., eds, Raven Press, New York, 1990, p. 1679; Berkner, K. L., Biotechniques 6:616 9191988), Strauss, S. E., In: The Adenoviruses, Ginsberg, H S, ed., Plenum Press, New York, 1984, chapter 11), herpes simplex virus (HSV) for neuron-specific delivery and persistence. Advantages of adenovirus vectors for human gene therapy include the fact that recombination is rare, no human malignancies are known to be associated with such viruses, the adenovirus genome is double stranded DNA which can be manipulated to accept foreign genes of up to 7.5 kb in size, and live adenovirus is a safe human vaccine organisms. Adeno-associated virus is also useful for human therapy (Samulski, R. J. et al., EMBO J. 10:3941 (1991) according to the present invention.

Another vector which can express the DNA molecule of the present invention, and is useful in the present therapeutic setting, particularly in humans, is vaccinia virus, which can be rendered non-replicating (U.S. Pat. Nos. 5,225,336; 5,204,243; 5,155,020; 4,769,330; Sutter, G et al., Proc. Natl. Acad. Sci. USA (1992) 89:10847-10851; Fuerst, T. R. et al., Proc. Natl. Acad. Sci. USA (1989) 86:2549-2553; Falkner F. G. et al.; Nucl. Acids Res (1987) 15:7192; Chakrabarti, S et al., Molec. Cell. Biol. (1985) 5:3403-3409). Descriptions of recombinant vaccinia viruses and other viruses containing heterologous DNA and their uses in immunization and DNA therapy are reviewed in: Moss, B., Curr. Opin. Genet. Dev. (1993) 3:86-90; Moss, B. Biotechnology (1992) 20: 345-362; Moss, B., Curr Top Microbiol Immunol (1992) 158:25-38; Moss, B., Science (1991) 252:1662-1667; Piccini, A et al., Adv. Virus Res. (1988) 34:43-64; Moss, B. et al., Gene Amplif Anal (1983) 3:201-213.

In addition to naked DNA or RNA, or viral vectors, engineered bacteria may be used as vectors. A number of bacterial strains including Salmonella, BCG and Listeria monocytogenes(LM) (Hoiseth & Stocker, Nature 291, 238-239 (1981); Poirier, T P et al. J. Exp. Med. 168, 25-32 (1988); (Sadoff, J. C., et al., Science 240, 336-338 (1988); Stover, C. K., et al., Nature 351, 456-460 (1991); Aldovini, A. et al., Nature 351, 479-482 (1991); Schafer, R., et al., J. Immunol. 149, 53-59 (1992); Ikonomidis, G. et al., J. Exp. Med. 180, 2209-2218 (1994)). These organisms display two promising characteristics for use as vaccine vectors: (1) enteric routes of infection, providing the possibility of oral vaccine delivery; and (2) infection of monocytes/macrophages thereby targeting antigens to professional APCs.

In addition to virus-mediated gene transfer in vivo, physical means well-known in the art can be used for direct transfer of DNA, including administration of plasmid DNA (Wolff et al., 1990, supra) and particle-bombardment mediated gene transfer (Yang, N.-S., et al., Proc. Natl. Acad. Sci. USA 87:9568 (1990); Williams, R. S. et al., Proc. Natl. Acad. Sci. USA 88:2726 (1991); Zelenin, A. V. et al., FEBS Lett. 280:94 (1991); Zelenin, A. V. et al., FEBS Lett. 244:65 (1989); Johnston, S. A. et al., In Vitro Cell. Dev. Biol. 27:11 (1991)). Furthermore, electroporation, a well-known means to transfer genes into cell in vitro, can be used to transfer DNA molecules according to the present invention to tissues in vivo (Titomirov, A. V. et al., Biochim. Biophys. Acta 1088:131 ((1991)).

“Carrier mediated gene transfer” has also been described (Wu, C. H. et al., J. Biol. Chem. 264:16985 (1989); Wu, G. Y. et al., J. Biol. Chem. 263:14621 (1988); Soriano, P. et al., Proc. Natl. Acad. Sci. USA 80:7128 (1983); Wang, C-Y. et al., Proc. Natl. Acad. Sci. USA 84:7851 (1982); Wilson, J. M. et al., J. Biol. Chem. 267:963 (1992)). Preferred carriers are targeted liposomes (Nicolau, C. et al., Proc. Natl. Acad. Sci. USA 80:1068 (1983); Soriano et al., supra) such as immunoliposomes, which can incorporate acylated mAbs into the lipid bilayer (Wang et al., supra). Polycations such as asialoglycoprotein/polylysine (Wu et al., 1989, supra) may be used, where the conjugate includes a molecule which recognizes the target tissue (e.g., asialoorosomucoid for liver) and a DNA binding compound to bind to the DNA to be transfected. Polylysine is an example of a DNA binding molecule which binds DNA without damaging it. This conjugate is then complexed with plasmid DNA according to the present invention for transfer.

Plasmid DNA used for transfection or microinjection may be prepared using methods well-known in the art, for example using the Quiagen procedure (Quiagen), followed by DNA purification using known methods, such as the methods exemplified herein.

FuGENE 6® Transfection Reagent (“FuGENE”) is a multi-component lipid-based reagent (Roche Molecular Systems) (non-liposomal formulation) that complexes with and transports DNA into a cell during transfection. See http://biochem.roche.com/prodinfo_fst.htm?/fugene/ where a

Benefits of FuGENE 6 Reagent include: very high transfection efficiency in many common cell types; virtually no cytotoxicity even in many primary cell types; functions exceptionally well in the presence or absence of serum and requires minimal optimization.

One day before the transfection, adherent cells are plated to a density that would yield around 50-80% confluence on the day of the experiment. For suspension cells, 10⁶ cells/ml are preferred. To transfect, add the appropriate amount of the FuGENE 6 to a serum-free medium. To this mixture, the DNA is added. After incubating for 15 minutes, the final DNA:FuGENE 6 mixture is added to the cells and the procedure is complete. The low cytotoxicity increases the number of cell types that may be transfected as well as the transfection efficiency. This approach eliminates the need to remove the reagent:DNA complex from the cells until one is ready to assay. Cells transfected with FuGENE 6 produce high levels of protein.

Therapeutic Compositions and Their Administration

The present invention contemplates any compound that inhibits the activity of AHSG in a mammalian subject, preferably a human. These are referred to collectively as “AHSG inhibitors.” An AHSG inhibitor may be a low molecular weight organic compound (a conventional “drug”) that interferes in one or another activity of AHSG that result in loss of its final action in promoting or inducing the autophosphorylation or the insulin-mediated phosphorylation, of IR.

Examples of levels at which AHSG may be inhibited include its expression (via mRNA synthesis, translocation or translation. These can be attacked by the use of antisense compositions or ribozymes (see above).

Because activated AHSG is phosphorylated at two Ser residues as described herein, and this state is required for AHSG action, then one embodiment of an AHSG inhibitor is a compound that blocks phosphorylation of these residues. An example is a protein kinase inhibitor, a number of which are know in the art. See, for example, Levitzki, A, Ernst Schering Res Found Workshop 2001;(34):71-80; Levitzki A., Med Oncol. June 1997;14(2):83-9; Levitzki A. Curr Opin Cell Biol. April 1996;8(2):239-44. Another embodiment is a phosphatase or other compound which dephosphorylates the key Ser residues of activated AHSG or promotes such dephosphorylation.

Another type of AHSG inhibitor is a compound which interferes with the AHSG action on IR-active TK's. Such a compound may block any required binding interactions between AHSG and the TK or the IR. Antibodies specific for AHSG, preferably mAbs, most preferably human mAbs would be expected to perform such functions.

An AHSG inhibitor as described herein is administered in a pharmaceutically acceptable carrier in a biologically effective or a therapeutically effective amount. The inhibitor may be given alone or in combination with another composition that is directed to treatment of the same disease or condition. The following doses and amounts also pertain to the antibodies of the invention when administered to a subject.

A therapeutically effective amount is a dosage that, when given for an effective period of time, achieves the desired metabolic or clinical effect.

A therapeutically active amount of an AHSG inhibitor (or an anti-AHSG antibody) may vary according to factors such as the disease state, age, sex, and weight of the individual, and the ability of the peptide to elicit a desired response in the individual. Dosage regimes may be adjusted to provide the optimum therapeutic response. For example, several divided doses may be administered daily or the dose may be proportionally reduced as indicated by the exigencies of the therapeutic situation.

Thus an effective amount is between about 1 ng and about 1 gram per kilogram of body weight of the recipient, more preferably between about 1 μg and 100 mg/kg, more preferably, between about 100 μg and about 100 mg/kg. Dosage forms suitable for internal administration preferably contain (for the latter dose range) from about 0.1 mg to 500 mg of active ingredient per unit. The active ingredient may vary from 0.5 to 95% by weight based on the total weight of the composition.

The active compound may be administered in a convenient manner, e.g., injection or infusion by a convenient and effective route. Preferred routes include subcutaneous, intradermal, intravenous and intramuscular routes. Other possible routes include oral administration, intrathecal, inhalation, transdermal application, or rectal administration.

Depending on the route of administration, the active compound may be coated in a material to protect the compound from the action of enzymes, acids and other natural conditions which may inactivate the compound. Thus, to a administer a polypeptide or peptide therapeutic by an enteral route, it may be necessary to coat the composition with, or co-administer the composition with, a material to prevent its inactivation. For example, a peptide may be administered to an individual in an appropriate carrier, diluent or adjuvant, co-administered with enzyme inhibitors (e.g., pancreatic trypsin inhibitor, diisopropylfluorophosphate (DEP) and trasylol).or in an appropriate carrier such as liposomes (including water-in-oil-in-water emulsions as well as conventional liposomes (Strejan et al., (1984) J. Neuroimmunol 7:27).

As used herein “pharmaceutically acceptable carrier” includes any and all solvents, dispersion media, coatings, antibacterial and antifungal agents, isotonic and absorption delaying agents, and the like. The use of such media and agents for pharmaceutically active substances is well known in the art. Except insofar as any conventional media or agent is incompatible with the active compound, use thereof in the therapeutic compositions is contemplated. Supplementary active compounds can also be incorporated into the compositions.

Preferred pharmaceutically acceptable diluents include saline and aqueous buffer solutions. Pharmaceutical compositions suitable for injection include sterile aqueous solutions (where water soluble) or dispersions and sterile powders for the extemporaneous preparation of sterile injectable solutions or dispersion. Isotonic agents, for example, sugars, polyalcohols such as mannitol, sorbitol, sodium chloride may be included in the pharmaceutical composition. In all cases, the composition should be sterile and should be fluid. It should be stable under the conditions of manufacture and storage and must include preservatives that prevent contamination with microorganisms such as bacteria and fungi. Dispersions can also be prepared in glycerol, liquid polyethylene glycols, and mixtures thereof and in oils. Under ordinary conditions of storage and use, these preparations may contain a preservative to prevent the growth of microorganisms.

The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants.

Prevention of the action of microorganisms can be achieved by various antibacterial and antifungal agents, for example, parabens, chlorobutanol, phenol, ascorbic acid, thimerosal, and the like.

Prolonged absorption of the injectable compositions can be brought about by including in the composition an agent which delays absorption, for example, aluminum monostearate and gelatin.

Parenteral compositions are preferably formulated in dosage unit form for ease of administration and uniformity of dosage. Dosage unit form refers to physically discrete units suited as unitary dosages for a mammalian subject; each unit contains a predetermined quantity of active compound calculated to produce the desired therapeutic effect in association with the required pharmaceutical carrier. The specification for the dosage unit forms of the invention are dictated by and directly dependent on (a) the unique characteristics of the active compound and the particular therapeutic effect to be achieved, and (b) the limitations inherent in the art of compounding such an active compound for the treatment of sensitivity in individuals.

For topical application, an AHSG inhibitor may be incorporated into topically applied vehicles such as salves or ointments as well as a means for administering the active ingredient directly. The carrier for the active ingredient may be either in sprayable or nonsprayable form. Non-sprayable forms can be semi-solid or solid forms comprising a carrier indigenous to topical application and having a dynamic viscosity preferably greater than that of water. Suitable formulations include, but are not limited to, solution, suspensions, emulsions, creams, ointments, powders, liniments, salves, and the like.

Other pharmaceutically acceptable carriers for the AHSG inhibitor according to the present invention are liposomes, pharmaceutical compositions in which the active component, e.g., protein, is contained either dispersed or variously present in corpuscles consisting of aqueous concentric layers adherent to lipidic layers. The active protein is preferably present in the aqueous layer and in the lipidic layer, inside or outside, or, in any event, in the non-homogeneous system generally known as a liposomic suspension. The hydrophobic layer, or lipidic layer, generally, but not exclusively, comprises phospholipids such as lecithin and sphingomyelin, steroids such as cholesterol, more or less ionic surface active substances such as dicetylphosphate, stearylamine or phosphatidic acid, and/or other materials of a hydrophobic nature.

Antibodies Specific for Epitopes of AHSG

In the following description, reference will be made to various methodologies known to those of skill in the art of immunology, cell biology, and molecular biology. Publications and other materials setting forth such known methodologies to which reference is made are incorporated herein by reference in their entireties as though set forth in full. Standard reference works setting forth the general principles of immunology include A. K. Abbas et al., Cellular and Molecular Immunology (Fourth Ed.), W. B. Saunders Co., Philadelphia, 2000; C. A. Janeway et al., Immunobiology. The Immune System in Health and Disease, Fourth ed., Garland Publishing Co., New York, 1999; Roitt, I. et al., Immunology, (current ed.) C. V. Mosby Co., St. Louis, Mo. (1999); Klein, J., Immunology, Blackwell Scientific Publications, Inc., Cambridge, Mass., (1990).

Monoclonal antibodies (mAbs) and methods for their production and use are described in Kohler and Milstein, Nature 256:495-497 (1975); U.S. Pat. No. 4,376,110; Hartlow, E. et al., Antibodies: A Laboratory Manual, Cold Spring Harbor Laboratory Press, Cold Spring Harbor, N.Y., 1988); Monoclonal Antibodies and Hybridomas: A New Dimension in Biological Analyses, Plenum Press, New York, N.Y. (1980); H. Zola et al., in Monoclonal Hybridoma Antibodies: Techniques and Applications, CRC Press, 1982)).

Immunoassay methods are also described in Coligan, J. E. et al., eds., Current Protocols in Immunology, Wiley-Interscience, New York 1991 (or current edition); Butt, W. R. (ed.) Practical Immunoassay: The State of the Art, Dekker, New York, 1984; Bizollon, Ch. A., ed., Monoclonal Antibodies and New Trends in Immunoassays, Elsevier, New York, 1984; Butler, J. E., ELISA (Chapter 29), In: van Oss, C. J. et al., (eds), IMMUNOCHEMISTRY, Marcel Dekker, Inc., New York, 1994, pp. 759-803; Butler, J. E. (ed.), Immunochemistry of Solid-Phase Immunoassay, CRC Press, Boca Raton, 1991; Weintraub, B., Principles of Radioimmunoassays, Seventh Training Course on Radioligand Assay Techniques, The Endocrine Society, March, 1986; Work, T. S. et al., Laboratory Techniques and Biochemistry in Molecular Biology, North Holland Publishing Company, NY, (1978) (Chapter by Chard, T., “An Introduction to Radioimmune Assay and Related Techniques”).

A preferred ELISA assay for AHSG is described in Example VIII herein.

Anti-idiotypic antibodies are described, for example, in Idiotypy in Biology and Medicine, Academic Press, New York, 1984; Immunological Reviews Volume 79, 1984; Immunological R̅eviews Volume 90, 1986; Curr. Top. Microbiol., Immunol. Volume 119, 1985; Bona, C. et al., CRC Crit. Rev. Immunol., pp. 33-81 (1981); Jerne, N K, Ann. Immunol. 125C:373-389 (1974); Jerne, N K, In: Idiotypes—Antigens on the Inside, Westen-Schnurr, I., ed., Editiones Roche, Basel, 1982, Urbain, J et al., Ann. Immunol. 133D:179-(1982); Rajewsky, K. et al., Ann. Rev. Immunol. 1:569-607 (1983)

The present invention provides antibodies, polyclonal and monoclonal, reactive with epitopes of AHSG, that are useful as AHSG inhibitors in vivo. The antibodies may be xenogeneic, allogeneic, syngeneic, or modified forms thereof, such as humanized or chimeric antibodies. Antiidiotypic antibodies specific for the idiotype of an anti-AHSG antibody are also included. The term “antibody” is also meant to include both intact molecules as well as fragments thereof that include the antigen-binding site and are capable of binding to a AHSG epitope. These include, Fab and F(ab′)₂ fragments which lack the Fc fragment of an intact antibody, clear more rapidly from the circulation, and may have less non-specific tissue binding than an intact antibody (Wahl et al., J. Nucl. Med. 24:316-325 (1983)). Also included are Fv fragments (Hochman, J. et al. (1973) Biochemistry 12:1130-1135; Sharon, J. et al.(1976) Biochemistry 15:1591-1594).). These various fragments are be produced using conventional techniques such as protease cleavage or chemical cleavage (see, e.g., Rousseaux et al., Meth. Enzymol., 121:663-69 (1986))

Polyclonal antibodies are obtained as sera from immunized animals such as rabbits, goats, rodents, etc. and may be used directly without further treatment or may be subjected to conventional enrichment or purification methods such as ammonium sulfate precipitation, ion exchange chromatography, and affinity chromatography (see Zola et al., supra).

The immunogen may comprise the complete AHSG protein, or fragments or derivatives thereof. Preferred immunogens comprise all or a part of the human AHSG, including residues contain the post-translation modifications, such as glycosylation, found on the native AHSG. Immunogens are produced in a variety of ways known in the art, e.g., expression of cloned genes using conventional recombinant methods, isolation from tissue of origin, expressing high levels of AHSG, etc.

The mAbs may be produced using conventional hybridoma technology, such as the procedures introduced by Kohler and Milstein (Nature, 256:495-97 (1975)),—and modifications thereof (see above references). An animal, preferably a mouse is primed by immunization with an immunogen as above to elicit the desired antibody response in the primed animal.

B lymphocytes from the lymph nodes, spleens or peripheral blood of a primed, animal are fused with myeloma cells, generally in the presence of a fusion promoting agent such as polyethylene glycol (PEG). Any of a number of murine myeloma cell lines are available for such use: the P3-NS1/1-Ag4-1, P3-x63-k0Ag8.653, Sp2/0-Ag14, or HL1-653 myeloma lines (available from the ATCC, Rockville, Md.). Subsequent steps include growth in selective medium so that unfused parental myeloma cells and donor lymphocyte cells eventually die while only the hybridoma cells survive. These are cloned and grown and their supernatants screened for the presence of antibody of the desired specificity, e.g. by immunoassay techniques using the AHSG-Ig fusion protein Positive clones are subcloned, e.g., by limiting dilution, and the mAbs are isolated.

Hybridomas produced according to these methods can be propagated in vitro or in vivo (in ascites fluid) using techniques known in the art (see generally Fink et al., Prog. Clin. Pathol., 9:121-33 (1984)). Generally, the individual cell line is propagated in culture and the culture medium containing high concentrations of a single mAb can be harvested by decantation, filtration, or centrifugation.

The antibody may be produced as a single chain antibody or scFv instead of the normal multimeric structure. Single chain antibodies include the hypervariable regions from an Ig of interest and recreate the antigen binding site of the native Ig while being a fraction of the size of the intact Ig (Skerra, A. et al. (1988) Science, 240: 1038-1041; Pluckthun, A. et al. (1989) Methods Enzymol. 178: 497-515; Winter, G. et al. (1991) Nature, 349: 293-299); Bird et al., (1988) Science 242:423; Huston et al. (1988) Proc. Natl. Acad. Sci. USA 85:5879; Jost C R et al,. J Biol Chem. 1994 269:26267-26273; U.S. Pat. Nos. 4,704,692, 4,853,871, 4,946,778, 5,260,203, 5,455,030.

The foregoing antibodies are useful in method for inhibiting AHSG activity and treating diseases or conditions associated with insulin resistance as discussed above. This method involves administering a subject in need of such treatment an effective amount of an antibody, preferably a mAb, more preferably a human or humanized mAb specific for an epitope of AHSG. The administration of antibody must be effective in blocking AHSG biological activity, such as insulin-stimulated IR phosphorylation. Relevant dose ranges are described elsewhere.

Having now generally described the invention, the same will be more readily understood through reference to the following examples which are provided by way of illustration, and are not intended to be limiting of the present invention.

### EXAMPLE I

### AHSG a Specific Inhibitor of Insulin Receptor Autophosphorylation, Interacts with the Insulin Receptor

This Example appears in a paper published by the present inventors and their colleagues in Mol Cell Endocrinol, 2000,164:87-98, which is incorporated by reference in its entirety.

Human AHSG inhibits the mitogenic pathway without affecting the metabolic arm of insulin signal transduction. This study described the time-course and specificity of inhibition, AHSG interaction with IR and probable physiological role. In intact rat fibroblasts overexpressing the human IR (HIRc B), incubation of recombinant human AHSG (1.8 μM) (“rhAHSG”) inhibited insulin-induced IR autophosphorylation by over 80%. This inhibitory effect of rhAHSG on insulin-induced IR autophosphorylation was blunted by half in 60 min. Interestingly, rhAHSG at similar concentrations (0.9 or 1.8 μM), had no effect on EGF- or IGF-I-induced cognate receptor autophosphorylation. Anti-AHSG immunoprecipitates of rhASHG-treated HIRc B cell lysates demonstrated the presence of IR. These results suggested that AHSG preferentially interacts with the activated IR.

To further characterize the site(s) of interaction, the effect of rhAHSG on trypsin-treated IR autophosphorylation was studied. Trypsin-treatment of intact HIRc B cells results in proteolysis of the IR α-chain and constitutive activation of IR-TK activity. The study demonstrate that rhAHSG (0.1 μM) completely inhibited trypsin-activated IR autophosphorylation and TK activity in vitro indicating that this effect was not mediated by its interaction with the proximal 576 amino acid residues of the IR α-subunit.

The physiological relevance of these observations was explored by characterizing the effects of AHSG injection in rats. RhAHSG (2 μM), acutely injected through the portal vein of normal rats, inhibited insulin-stimulated IR autophosphorylation and IRS-1 phosphorylation in liver and hindlimb muscle. Taken together these results showed that AHSG, by interacting with IR, specifically inhibits insulin-stimulated IR autophosphorylation and plays a physiological role in the regulation of insulin signaling.

### EXAMPLE II

### Materials Methods for Examples III-VII

Animals

Double homozygous Ahsg KO (Ahsg^{-/-}) mice from a mixed background (Jahnen-Dechent, W. et al., J Biol Chem 272, 31496-31503 (1997))²⁷ were backcrossed four generations into C57B1/6J. Offspring [Ahsg KO and WT littermates (Ahsg^{+/+})] from the fourth generation of this breeding protocol were used for this study. Mice were housed on a 12-hour light/dark cycle and fed a standard rodent chow. All protocols for animal use and euthanasia were reviewed and approved by the Animal Investigation Committee of Wayne State University in accordance with NIH guidelines. For in vivo studies, animals were anesthetized with ketamine (80 mg/kg) and xylazine (5 mg/kg) IP, and insulin (0.1, 1 and 10 μM) was injected through the portal vein. Saline-injected animals served as controls. Liver and hindlimb muscles were excised 1 and 3 min later, respectively, as described earlier (Saad, M. J. A. et al., J Clin Invest 90, 1839-1849 (1992))⁵. Surgical procedures: Mice were anesthetized with an injection of pentobarbital (65 mg/g body weight i.p) and an indwelling catheter was implanted as described by others (Kamohara, S. et al., Nature 389, 374-377 (1997))⁵⁶. Briefly a 5-mm incision was made on the ventral side of the left leg at the level of the hip. A catheter was inserted into the femoral vein and secured. The catheter was sealed subcutaneously and exteriorized at the base of the neck. Mice underwent a 2-day observation period and those exhibiting signs of illness were excluded from the study (2 animals). High fat feeding: Forty-three female mice were used to study the effect of high fat diet on body-weight gain and insulin sensitivity. Mice were housed 4-5 per hanging cage with food and water available ad-libitum. Within each genotype (KO and WT), they were divided into high fat (HF) and low fat (LF) fed groups. The LF diet was based on AIN-93M formula (Reeves, P. G. et al., J Nutr 123, 1939-1951 (1993))⁵⁷ with 4% fat in the form of soybean oil. The HF diet was a modification of AIN-93M formula with added soybean oil so the final fat content was 40% by weight. The caloric content of these two diets for carbohydrate, protein and fat were: 75.9%, 14.1% and 10% for LF diet and 26.17%, 15.06% and 58.77% for HF diet. Diets were prepared by Dyets, Inc. (Bethlehem, Pa.) and stored in cold room until use. WT and KO mice were fed HF or LF diet for a period of 9 weeks. A known amount of fresh food was offered to mice twice per week in a double-jar setup to reduce spillage. Food intake and body weight were measured once a week. Food left in the jar was weighed after spillage was collected. For body composition analysis, internal organs were dissected out and all visible internal fat was removed and weighed. The remaining carcass was frozen for carcass analysis (Jen, K.-L. C. et al., Physiol Behav 27, 161-166 (1981))⁵⁸. In brief, the carcass was shaved, autoclaved and homogenized with distilled water using a polytron homogenizer (Brinkmann, Westbury, N.Y.). The carcass fat, designated subcutaneous fat, was extracted by the method of Folch et al. (Folch, J. et al., J Biol Chem 226, 497-509 (1957))⁵⁹. The sum of subcutaneous fat and internal fat was the total body fat for each mouse.

Partial Purification of IR, Autophosphorylation and TK Activity

IR were partially purified on wheat germ agglutinin (WGA)-agarose columns and eluted with 0.3M N-acetylglucosamine. IR autophosphorylation of the partially purified IR, in the presence or absence of insulin, was carried out by the addition of (γ³²-P) ATP to a reaction mixture containing 5 mM MnCl₂, 50 μM ATP. 50 mM HEPES, pH 7.6 and 0.1% Triton X-100 and the proteins were then separated on 7.5% SDS-PAGE. IR-TK activity was assayed by quantitation of phosphorylation on exogenous substrate, poly (Glu⁸⁰Tyr²⁰), as described earlier Mathews et al., supra (2000)²¹.

Metabolic Studies

For glucose tolerance tests, an oral (1 mg/g body weight) or intra-peritoneal (1.5 mg/g body weight) glucose load was given after a 16-hour fast, to 10-week old, male or female wild type and Ahsg KO mice. Blood samples were taken at 0, 15, 30, 60 and 120 min from the tail vein. Glucose levels were measured with a Glucometer Elite blood glucose monitor (Bayer, Elkhart, Ind.). For insulin tolerance test, random-fed female mice, all 10 weeks of age, were given an intra-peritoneal injection of 0.75 or 0.15 U/kg body weight regular human insulin (Novolin R) (Novo Nordisk, Clayton, N.C.) between 2:00 and 5:00 P.M. Blood samples were obtained at various time points from the tail vein and glucose levels were measured as described above. Insulin levels were measured in plasma using commercial radioimmunoassay kits (Linco Research Inc., St. Charles, Mo.) using rat insulin standards. To assess lipid levels, blood samples were obtained by retro-orbital bleeds from overnight fasted anesthetized mice. Fasting triglyceride levels (TG) were measured in plasma by a colorimetric assay (Sigma) and fasting free fatty acid (FFA) concentrations were determined using the NEFA C kit (Wako Chemicals USA, Richmond, Va.). Fasting levels of leptin were assayed with a mouse leptin RIA kit (Linco Research Inc.,).

Euglycemic-Hyperinsulinemic Clamp

Clamp studies were carried out on five male KO mice (3-4 months old) and five, age- and sex-matched WT mice, as previously described (Kamohara, S. et al., Nature 389, 374-377 (1997); Massillon, D. et al., Am J Physiol 269, E1037-43 (1995)^{56, 60}. Food was removed 5-6 hours prior to infusion. A bolus of 3-[³H]-glucose (50 μCi) was administered at the start of each clamp over a 1 minute time period. For the remainder of the clamp, 3-³H-glucose was infused at 12 μCi/Kg/min. A continuous infusion of porcine insulin (Eli Lilly, Indianapolis, Ind.) was administered at 100 mU/min/Kg. Plasma glucose was clamped at 90-110 mg/dL by infusing a 20% glucose solution. Glycemia was assessed on blood obtained from the tail vein using a One Touch II Glucose Meter (LifeScan, Milpita, Calif.). Steady state glucose levels were achieved after approximately 80 minutes at which point 10 μl of blood was collected every 10 minutes for 40 minutes. The animals were then given a bolus (24 μCi) of [¹⁴C]-2-deoxyglucose (2-DOG), which was flash-injected through the catheter and 10 μl of blood was collected at 2, 4, 6, 8, 10, 20, 30 and 40 minutes. At the end of the 40-minute period, the animals were sacrificed. Tissues (brown adipose, heart, diaphragm, soleus, extensor digitorum longus (EDL), gastrocnemius, skin and white adipose) were rapidly removed and snap frozen in liquid nitrogen for further analysis. Whole body glucose utilization and tissue 2-DOG uptake were calculated as previously described^{56, 60}. Muscle glycogen content was determined by the amyloglucosidase method as previously described (Burcelin, R. et al., Diabetologia 38, 283-290 (1995))⁶¹.

Antibodies

Antibodies against insulin receptor β-subunit, phosphotyrosine proteins (4G10) and ERK2 were purchased from Upstate Biotechnology (Lake Placid, N.Y.). p44/42 MAP kinase assay kit, phospho-p44/42 MAP kinase antibody and phospho-Akt antibody were purchased from New England Biolabs (Beverly, Mass.).

Immunoprecipitations and Immunoblotting

Liver and muscle tissues were excised and homogenized in ice cold buffer A (50 mM HEPES, pH 7.4, 25 mM NaPPi, 100 mM NaF, 10 mM EDTA, 10 mM Na₃VO₄, 2 mM phenylmethylsulfonyl fluoride (PMSF), 1% Triton X-100, 10 μg/ml aprotinin and leupeptin). Immunoprecipitations were carried out overnight at 4° C. with required antibodies followed by addition of protein A and G sepharose beads (Oncogene, Cambridge, Mass.) for another hour at 4° C. Immunoprecipitated proteins (IR-β subunit, phosphorylated p44/42 MAPK) were washed, boiled in SDS-sample buffer and separated on 7.5% SDS-PAGE, transferred to nitrocellulose membrane (Schleicher and Schuell, Keene, N.J.) and developed using appropriate combinations of primary/secondary antibodies and chemiluminescence. Phosphorylation status of MAP kinase and Akt was assayed by Western blotting using phospho p44/42 MAPK antibody and phospho Akt antibody respectively. Quantitation of ERK2, IR-β subunit and Akt-1 were done to normalize the phosphorylation data to protein loading. MAPK activity was assayed using a kit with two phospho-specific antibodies (New England Biolabs, Beverly, Mass.). Briefly, activated MAPK was selectively precipitated using phospho p44/42 antibody (Thr202 and Tyr204). The resulting immunoprecipitate was incubated with a Elk-1 fusion protein in the presence of ATP and kinase buffer, which allows active MAP kinase to phosphorylate Elk-1. Phosphorylation of Elk-1 was then measured by Western blotting using a phospho-Elk-1 (Ser383) antibody.

Statistical Analysis

Data are presented as mean±SEM. Statistical analyses (Student's t-test or Analysis of Variance (ANOVA) were performed using “GraphPad Instat” (San Diego, Calif.). Differences were considered significant if P≦0.05. Quantitation of data from Western blots and autoradiographs were done using “UN-SCAN-IT Gel Automated Digitizing System” (Silk Scientific, Orem, Utah).

### EXAMPLE III

### Increased Insulin Receptor (IR) Autophosphorylation and Tyrosine Kinase (TK) Activity

Since AHSG inhibits insulin-induced IR autophosphorylation and TK activity it was predicted that genetic ablation of AHSG would result in increased insulin-induced IR autophosphorylation and TK activity. To verify this, the present inventors examined both basal and insulin-induced IR autophosphorylation status in vitro (partially purified IR) and in vivo (liver and skeletal muscle).

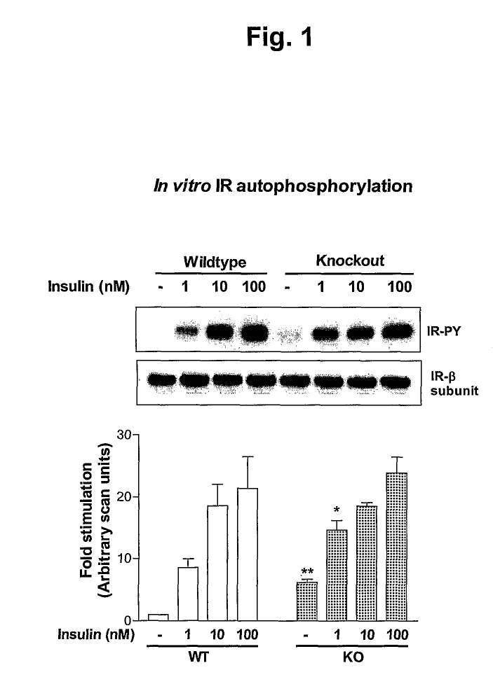

IRs were partially purified by wheat germ agglutinin column chromatography from livers of age-, weight- and sex-matched KO and WT mice. IR autophosphorylation and TK activity were studied in vitro. A representative autoradiograph (from 4 separate experiments with IRs purified individually from livers of WT and KO mice, n=4 mice per group) of in vitro IR-β subunit autophosphorylation is illustrated (FIG. 1, upper panel). AHSG KO mice showed ˜4-fold increase in basal IR autophosphorylation compared to WT mice.

Insulin-induced IR autophosphorylation was increased in KO mice compared to WT. The extent of IR-β subunit phosphorylation induced by 1 nM insulin in KO mice was higher (14.26±1.55 fold stimulation over WT basal-arbitrary scan units: FIG. 1, bar diagram) compared to WT mice (8.56±1.38 arbitrary scan units). Insulin-induced IR autophosphorylation was similar at higher insulin concentrations (10 or 100 nM) in WT and KO mice. Western blotting with an antibody against insulin receptor β-subunit confirmed equal amounts of IR loading in both WT and KO lanes (FIG. 1, bottom panel).

TK activity was assayed in vitro in WGA-purified IR from KO and WT mice. Basal TK activity was significantly increased (p<0.001) in IR from KO mice (FIG. 2), analogous to results of receptor autophosphorylation.

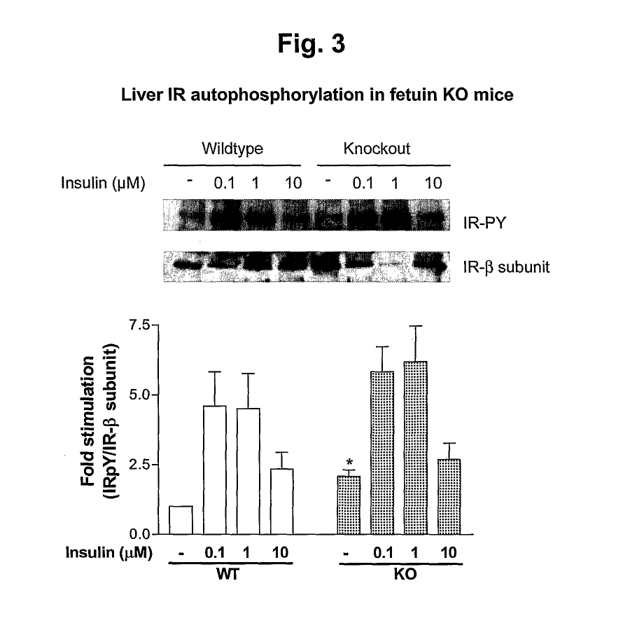

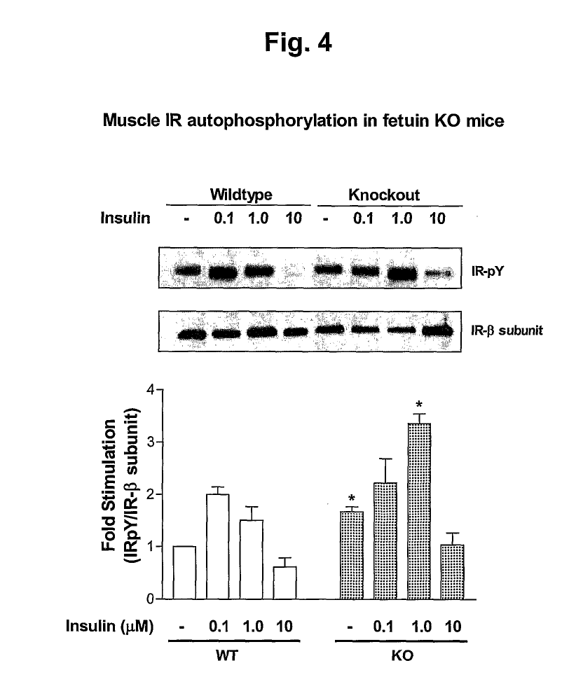

Next, autophosphorylation of IR-β subunit in liver and skeletal muscle, after in vivo exposure to insulin (portal vein injection of 0.1, 1 or 10 μM insulin), was assayed in age-, weight- and sex-matched WT and KO mice. Saline-injected mice served as controls. Representative blots of IR autophosphorylation in liver (FIG. 3, panel 1) and skeletal muscle (FIG. 3, panel 1) are depicted. IR phosphorylation data in liver and muscle were normalized to IR β-subunit levels (FIG. 3 panel 2; FIG. 4, panel 2) and the combined data from 4 separate experiments are shown as bar diagrams. A two-fold increase in basal IR autophosphorylation in liver (p<0.05) and ˜1.5-fold increase (p<0.05) in basal IR phosphorylation in skeletal muscle were observed in Ahsg KO mice. A significant increase (p<0.05) in insulin-induced (1 μM) IR phosphorylation was observed in skeletal muscle of KO mice, and a similar increasing trend in insulin-induced IR phosphorylation was observed in livers from KO mice.

### EXAMPLE IV

### Increased MAPK and Akt Phosphorylation

To confirm that the increased phosphorylation of IR in liver and muscle of Ahsg KO mice is manifested in increased downstream signaling, phosphorylation status of p44/42 MAPK and Akt were assayed following in vivo exposure to insulin (portal vein injection of 0.1, 1 or 10 μM insulin) or saline in age-, weight- and sex-matched WT and KO mice. In liver, phosphorylation of MAPK was assayed by phospho-p44/42 MAPK antibody and its activity by detecting MAPK-induced phosphorylation of Elk-1. In livers of KO mice, basal phosphorylation of p44/42 MAP kinase was increased ˜2 fold (FIG. 5, panel 1). Injection of insulin through the portal vein induced ˜5 fold increase in p44/42 MAPK phosphorylation compared to WT mice for every dose of insulin tested. Reprobing the membrane with ERK2 antibody confirmed equal sample loading (FIG. 5, panel 2).

MAPK activity assayed in liver homogenates (by active MAP kinase phosphorylation of Elk-1) demonstrated increased basal and insulin-stimulated phosphorylation of phospho-Elk-1, in concurrence with p44/42 MAPK phosphorylation (data not shown).

Similarly, both basal and insulin-induced phosphorylation of Akt, measured by phospho-Akt antibody, was increased in liver homogenates of Ahsg KO mice when compared to WT mice (FIG. 5, panel 3). Equal loading was confirmed using an antibody against Akt-1 (FIG. 5, panel 4). A representative blot (from 4-5 separate experiments) for each protein is depicted (FIG. 5).

In skeletal muscle, all tested doses of insulin induced greater amounts of p44/42 MAP kinase phosphorylation in Ahsg KO mice compared to WT mice (FIG. 6, panel 1). Equal loading of all lanes was confirmed using an antibody against ERK2 (FIG. 6, panel 2). Insulin-induced phosphorylation of Akt, measured by phospho-Akt antibody, was also increased in muscle (FIG. 6, panel 3) of Ahsg KO mice compared to WT mice. Equal loading was confirmed by nearly similar concentrations of Akt-1 (FIG. 6, panel 4). Basal phosphorylation of both MAPK and Akt was increased in skeletal muscle of KO mice. A representative blot (from 4-5 separate experiments) for each protein is depicted (FIG. 6).

Taken together, these results confirmed the present inventors prediction that genetic ablation of AHSG increases basal and insulin-induced IR autophosphorylation and TK activity. The increased basal and insulin-induced IR phosphorylation is reflected in the observed increase in phosphorylation of downstream signaling molecules (MAPK and Akt), suggestive of increased insulin sensitivity in Ahsg KO mice.

### EXAMPLE V

### Enhanced Glucose Clearance and Increased Insulin Sensitivity in Ahsg KO Mice

Since Ahsg KO mice demonstrated increased insulin signaling, glucose clearance rates were examined by glucose and insulin tolerance tests in 8-10 weeks old KO and WT mice. Ahsg KO mice cleared postprandial glucose from the blood with an increased efficiency over wild-type (WT) mice during oral glucose tolerance tests (GTT) (1 mg/g body weight). Differences between WT and KO mice blood glucose values were statistically significant p<0.01) at the 15, 30 and 60 min time points (FIG. 7a). This experiment was repeated twice using different sets and gender of animals (n=6/group) with similar results.

Though Ahsg KO mice, on average, have significantly lower body weights (p=0.005) compared to age- (10 weeks old) and sex-matched controls [Table 1; Female mice—WT: 15.5±1.2 g (n=14) vs. KO: 12.2±0.9 g (n=17; p<0.001)], the body weights for KO and WT mice undergoing the oral GTT were not statistically different (WT: 17.54±2.21 g; KO: 15.84±1.51 g, p=0.06) since the body weights of some WT and KO mice overlap.

However, to exclude the possibility of body weight affecting the outcome of the test, oral GTT was also done on age-, sex- and weight-matched mice (WT: 16.44±0.09 g, n=5; KO: 16.40±0.28 g, n=5) with similar results (FIG. 7b).

Further, GTT was also done with an intra-peritoneal injection of glucose (1.5 mg/g body weight; mean body weights—WT: 22.26 g; KO: 22.77 g) to exclude the possibility of defective gastrointestinal absorption or absorption-related mechanisms. Similar to oral GTT, diverging curves were obtained for the intra-peritoneal GTT, with KO mice displaying significantly enhanced glucose disposal compared to WT mice (p<0.05 for 15 min time-point and p<0.01 for 30 and 60 min time-points) (FIG. 7c).

Plasma insulin concentrations, after an intra-peritoneal glucose load, showed identical responses in KO and WT mice (FIG. 7d). The observed enhanced glucose disposal and normal insulin levels after a glucose load suggest an enhanced sensitivity to insulin in the KO mice.

To examine this possibility, insulin tolerance tests (ITT) were done on 8-10 weeks old, male WT and KO animals fed ad libitum. Using a single i.p. injection of regular human insulin (Novolin ®) of 0.75 U/kg body weight, no differences were obtained in the clearance of glucose from the blood of WT and KO mice (FIG. 7e). However, when a lower dose of insulin was injected (0.15 U/kg body weight), the difference in decrease of blood glucose levels between KO and WT mice was statistically significant (p<0.05): ˜45% drop in blood glucose in KO mice at 30 min, compared to 30% in the WT (FIG. 7f).

These results indicated that mice completely deficient for AHSG show markedly enhanced glucose handling and increased sensitivity to insulin action.

Fasting or fed (random) blood glucose or plasma insulin levels are not altered in male (Table 1) or female KO mice.

Since insulin resistance is associated with increased levels of plasma free fatty acids (FFA) and triglycerides (TG), it was predicted that plasma concentrations of free fatty acids and triglycerides would be decreased in Ahsg KO mice compared to WT mice. Ahsg KO mice demonstrate significantly lower levels of plasma FFA and TG (p=0.001) under fasting conditions, compared to WT mice (Table 1). Fasting levels of plasma leptin were not significantly altered in Ahsg KO mice compared to WT mice (Table 1). Levels of FFA, TG and leptin were assayed only in females due to blood sample limitations.

TABLE 1
Body weight, blood and plasma measurements
in 10-week old KO and WT mice.

| | Wild-Type | Knock-out | P value |
|---|---|---|---|
| Body-weight (g) | 17.9 ± 0.8 | 13.9 ± 0.9 | 0.005 |
| | (11) | (10) | |
| Fasting Blood | 79.8 ± 5.9 | 98.3 ± 7.7 | 0.245 |
| Glucose (mg/dL) | (9) | (10) | |
| Fed Blood | 120.2 ± 6.4 | 107.0 ± 10.0 | 0.272 |
| Glucose (mg/dL) | (11) | (10) | |
| Fasting Insulin | 0.177 ± 0.01 | 0.191 ± 0.01 | 0.337 |
| (ng/mL) | (11) | (10) | |
| Fed Insulin | 0.213 ± 0.02 | 0.187 ± 0.02 | 0.448 |
| (ng/mL) | (7) | (8) | |
| Fasting Free | 0.827 ± 0.06 | 0.599 ± .03 | 0.001 |
| Fatty Acids | (11) | (11) | |
| (mEq/L) | | | |
| Fasting | 59.52 ± 1.71 | 42.31 ± 4.33 | 0.001 |
| Triglycerides | (12) | (11) | |
| (mg/dl) | | | |
| Fasting Leptin | 1.33 ± 0.07 | 1.63 ± 0.46 | 0.068 |
| (ng/mL) | (12) | (12) | |

Values are mean ± S.E.M. Figures in parentheses indicate number of animals.

### EXAMPLE VI

Increased Whole Body Glucose Utilization and 2-Deoxyglucose Uptake

Euglycemic (100 mg/dL) clamps were performed on male Ahsg KO mice and age-, sex- and weight-matched WT controls to assess glucose utilization under hyperinsulinemic (100 mU/min/kg) conditions. Glucose infusion rates (GIR) in Ahsg KO were higher (113.8±6.4 mg/kg/min) than those measured in WT control mice (94.4±7.1 mg/kg/min) (p=0.077) (FIG. 8a). No differences in plasma insulin and plasma glucose were detected among KO and WT mice (data not shown). During the last 40 minutes of the euglycemic clamp a bolus of [¹⁴C]-2-deoxyglucose (2-DOG) was administered to determine glucose uptake rates in individual tissues. Although no significant differences were identified between Ahsg KO and control mice in the tissues sampled, an increasing trend was observed in 2-DOG uptake in soleus muscle, gastrocnemius and white adipose tissue of KO mice (FIG. 8b).

To assess the fate of glucose under insulin action, glycogen content was measured in heart, hindlimb, and liver at the end of the hyperinsulinemic clamp study. Hindlimb glycogen content was ˜1.9 fold greater in Ahsg KO mice (FIG. 8c), which is consistent with the increase in 2-DOG uptake. No significant differences were measured in heart and liver glycogen content between groups.

### EXAMPLE VII

### Obesity Resistance in Ahsg-Null Mice

HF feeding induces body weight gain and obesity (Jen, K.-L. C. Physiol Behav 42, 551-556 (1988); Jen, K.-L. C. et al., Int J Obes 19, 699-708 (1995))^{29, 30} and is associated with insulin resistance (Buchanan, T. A. et al., Am J Physiol 263, R785-789 (1992); Storlien, L. H. et al., Am J Physiol 251, E576-583 (1986))^{31, 32}.

Since Ahsg KO mice demonstrate increased insulin sensitivity, a study was performed to test if a HF diet would induce body weight gain and insulin resistance in these KO mice. Ahsg KO and WT mice (females, 10 weeks old) were fed HF (58.77% of calories from fat) or LF diet ad libitum for 9 weeks and monitored weekly for food intake. Body weight parameters at the end of study, total caloric intake, fasting blood glucose and plasma insulin concentrations are shown in Table 2 in which results are expressed as mean±S.E.M. Numbers with different superscripts are significantly different from each other, based either on genotype or diet.

At the end of the 9-week period, WT mice on HF diet had significantly higher body weight (p<0.005) compared to WT mice on LF diet (Table 2).

Remarkably, KO mice (at 9 weeks), remained lean with body weights comparable to WT mice on LF diet. In WT mice, the HF diet produced a 15.83% increase in body weight. Ahsg KO mice were substantially protected from diet-induced weight gain with an average increase in body weight of only 8.44%. The total caloric intake (over 9 weeks) by WT and KO mice was not different (6045±180 kcal for WTHF vs. 5652±499 kcal for KOHF). The weight to length ratio was significantly higher in WTHF mice (p<0.01) compared to KOHF mice. Total fat weight was significantly higher in WTHF mice compared to KOHF mice (p<0.01). Similar results were obtained when expressed as percent total fat (ratio of total fat weight to body weight), with KOHF mice showing significantly lower percentage (p<0.01) of total fat compared to WTHF mice.

The effect of HF diet on insulin levels and insulin sensitivity was assessed in Ahsg KO and WT mice. WTHF mice showed significantly higher fasting insulin levels (p<0.05) compared to KOHF mice (Table 2). Fasting blood glucose concentrations did not differ among the four groups of mice. In response to an intra-peritoneal glucose load (1.5 mg/g body weight i.p.), no impairment in glucose tolerance was observed in any of the four groups (data not shown). Though the secretory response of insulin was not altered, insulin levels at zero time and at 30 and 60 min time-points after the GTT were significantly higher in WTHF compared to KOHF mice (FIG. 9). Using the homeostasis model assessment (HOMA) of insulin sensitivity

$$\frac{( fasting glucose [ mmol \text{/} l ] \times fasting insulin [ μU \text{/} ml ]}{22.5}$$

(Matthews, D. R. et al., Diabetologia 28, 412-419 (1985)), KOHF mice demonstrated HOMA scores similar to WTLF mice, indicating that they (KOHF mice) retained their insulin-sensitivity (FIG. 10) while WTHF mice showed significantly higher HOMA scores (p<0.05), reflecting insulin resistance.

TABLE 2
Body Weight Parameters, Blood Glucose and Plasma Insulin in WT and KO Mice Fed an LF or HF Diet

| | WT-HF | WT-LF | KO-HF | KO-LF | | |
|---|---|---|---|---|---|---|
| | (n = 11) | (n = 11) | (n = 11) | (n = 10) | Genotype | Diet |
| Body wt (g) | 28.1 ± 0.9^{a} | 24.3 ± 0.9^{b} | 24.4 ± 1.0^{b} | 22.5 ± 0.9^{b} | p < 0.01* | P < 0.005 |
| Wt/Lgth (g/cm) | 2.85 ± 0.09^{a} | 2.55 ± 0.07^{b} | 2.5 ± 0.1^{b} | 2.39 ± 0.08^{b} | p < 0.01 | p < 0.05 |
| Liver wt (g) | 1.07 ± 0.04 | 1.1 ± 0.04 | 1.00 ± 0.04 | 1.07 ± 0.03 | ns | ns |
| Total Fat wt (g) | 5.8 ± 2.1^{a} | 3.4 ± 1.9^{b} | 3.7 ± 2.1^{b} | 2.62 ± 1.1^{b} | p < 0.01 | p < 0.005 |
| Total Fat % | 20.1 ± 0.6^{a} | 13.5 ± 0.6^{b} | 14.4 ± 0.6^{b} | 11.4 ± 0.4^{b} | p < 0.01 | p < 0.005 |
| Food Intake | 6045 ± 180^{a} | 3505 ± 36^{b} | 5652 ± 499^{a} | 3713 ± 229^{b} | ns | p < 0.01 |
| (kcalories) | | | | | | |
| Fasting Glucose | 93.64 ± 7.1 | 83.64 ± 7.2 | 84.91 ± 3.9 | 80.8 ± 4.8 | Ns | Ns |
| (mg/dl) | | | | | | |
| Fasting | 0.36 ± 0.03³ | 0.32 ± 0.01 | 0.28 ± 0.01^{b} | 0.30 ± 0.01 | p < 0.05 | ns |
| Insulin(ng/ml) | (n = 8) | (n = 9) | (n = 9) | (n = 8) | | |

*p values indicating statistical significance;
ns—not significant

### Discussion of Results in Examples II-VII

Decreased action of insulin in peripheral tissues is a central feature of several common pathological states including type 2 diabetes, obesity, hypertension and glucocorticoid excess (Reaven, supra; Kahn, C. R. Diabetes 43, 1066-84 (1994)). Although genetic defects in IR itself are rare, decreases in IR number and TK activity in muscle and other tissues of rodents and humans with early type 2 diabetes have been documented (Kahn, C. R. et al., J Biol Chem 248, 244-250 (1973); Bar, R. S. et al., J Clin Invest 58, 1123-1135 (1976); Kolterman, O. G. et al., J Clin Invest 68, 957-69 (1981); Prince, M. J. et al., Diabetes 30, 596-600 (1981); Grunberger, G. et al., Science 223, 932-934 (1984); Comi, R. J. et al., J Clin Invest 79, 453-467 (1987)). Improving insulin sensitivity offers a promising approach to the prevention, intervention and/or treatment of these pathological conditions. Several animal models demonstrate a potential to increase insulin sensitivity e.g., Pik3r1^{-/-}, PPARγ^{±}, PTP1B Ex1^{-/-} and transgenic Ucp-L mice (Terauchi, Y. et al., Nat Genet 21, 230-235 (1999); Kubota, N. et al., Mol Cell 4, 597-609 999); Elchebly, M. et al., Science 283, 1544-1548 (1999); Klaman, L. D. et al., Mol Cell Biol 20, 5479-5489 (2000); Li, B. et al., Nat Med 6, 1115-1120 (2000)). AHSG has “irstatin” (IR inhibitory) activity and interacts with the activated IR (Auberger et al., supra; Mathews et al., 2000, supra); Srinivas, P. R. et al., Cell Signal 8, 567-73 (1996)). In this study, we used mice that carry two null alleles for the Ahsg gene to examine the hypothesis that deficiency of AHSG leads to increased IR autophosphorylation and downstream insulin signaling, thereby improving whole body insulin sensitivity.

Ahsg KO mice exhibit increased insulin sensitivity, as evidenced by augmented phosphorylation of IR, TK activity, activation of MAP kinase and Akt and enhanced glucose clearance rates. Both in vitro and in vivo studies demonstrate increased IR autophosphorylation in muscle and liver of Ahsg KO mice. The increased basal TK activity of partially purified IR reflects in vivo IR phosphorylation status. The observed increase in basal IR phosphorylation (no added insulin) and TK activity and moderate increases in insulin-stimulated IR autophosphorylation in KO mice validates the irstatin role of AHSG. The increased insulin-stimulated signaling of downstream molecules (e.g., MAPK and Akt) in KO mice also implicates increased IR activation. The discrepancy of decreased IR phosphorylation at the highest insulin concentrations (10 μM) maybe due to IR down regulation after in vivo insulin exposure and/or due to dose/time dependent effects. It may be noted that the observed dose-dependent variations are similar in both WT and KO mice.

While the changes in insulin responsiveness, ranging from mild to moderate may be due to the significant reduction in the body weight of the KO mice, this is unlikely because weight-matched animals were used in several of the above experiments. The observed reduction in body weight may be due to decreased fat stores resulting from altered lipid metabolism and/or increased energy expenditure. Interestingly, other mouse models of increased insulin sensitivity such as PTP1B knockout mice, PPARγ heterozygous mice and mice that lack the Klotho gene also show significant reduction in size (Kubota et al., supra; Elchebly et al., supra; Klaman et al., supra; Mori, K. et al. Biochem Biophys Res Commun 278, 665-670 (2000)). On the contrary, mice that are selectively insulin resistant in muscle have an obese phenotype (Kim, J. K. et al., J Clin Invest 105,1791-743 (2000)).

Oral and intraperitoneal GTT and ITT demonstrate increased glucose clearance and improved insulin sensitivity in Ahsg KO mice. However, fasting glucose levels are not significantly altered. The fact that Ahsg KO mice showed an enhanced glucose clearance was surprising since previous findings show that AHSG only inhibited insulin's mitogenic effects without affecting insulin's metabolic effects (glycogen synthesis, amino acid uptake, tyrosine amino transferease activity) (Srinivas et al., supra, 1993)). If only 2-5 percent IR occupancy is required to exert such physiological effects as full mobilization of glucose transport (Sung, C. K. J Cell Biol 48, 26-32 (1992); Simpson, I. A. & Hedo, J. A., Science 223, 1301-1304 (1984)), then a small “leakage” resulting from AHSG's TK inhibition could potentially still drive the metabolic arm of insulin signaling. Whether the earlier studies reflect results of incomplete inhibition or use of less insulin-sensitive cell types, additional studies are required to clarify this point. Improved insulin sensitivity has been shown to be associated with decreased fasting insulin levels and decreased insulin secretion in response to a glucose challenge (Terauchi, Y. et al., supra; Leturque, A. et al., Diabetes 45, 23-27 (1996); Tsao, T.-S. et al., Diabetes 45, 28-36 (1996)). However, Ahsg KO mice do not show any difference in fasting or fed insulin levels. In response to a glucose load, insulin levels are marginally lower but not statistically significant. Ahsg KO mice demonstrate increased insulin sensitivity, as assessed by ITT, only at lower concentrations of insulin (0.15 U/kg body weight). This increased sensitivity of Ahsg KO mice at low insulin concentrations may be metabolically meaningful considering the fact that basal IR phosphorylation is elevated in KO mice. Further, it is possible that the insulin sensitivity is masked at higher insulin concentrations.

Under euglycemic-hyperinsulinemic clamp conditions, whole body glucose disposal was increased in KO mice, almost reaching statistical significance (p=0.077, n=5). Though 2-DOG uptake into muscle of KO mice only showed a trend towards increased insulin effect compared to WT mice, the glycogen content of hindlimb muscles was increased significantly in the KO mice after the hyperinsulinemic clamp study, indicating an increased shunting of infused glucose to glycogen in muscle (liver glycogen content was not altered in KO mice compared to WT mice). These data support the increased glucose clearance rates observed during oral and intra-peritoneal GTT. However, the possibility of alterations in glycogen breakdown as a cause of increased skeletal muscle glycogen content cannot be discounted.

Since an increased insulin sensitivity and lowered plasma lipid content was observed in Ahsg KO mice, consistent with improved insulin sensitivity, it was hypothesized that a high-fat diet would lead to less insulin resistance and body-weight gain in KO mice compared to WT mice. As expected, in WT mice, HF-feeding induced higher body weight compared to WTLF mice. However, KO mice responded to HF feeding differently; KOHF mice weighed as much as KOLF mice. The total body fat content was significantly lower in KOHF mice compared to WTHF mice. Further, KO mice maintained insulin sensitivity even after 9 weeks of HF feeding, unlike WTHF mice that became hyperinsulinemic and less insulin sensitive. Since KO mice lack AHSG, IR autophosphorylation can proceed more effectively, thus presumably maintaining normal glucose metabolism in face of HF feeding. Whether the resistance to weight gain is related to increased insulin sensitivity per se, increased basal metabolic rate (BMR) or other non-BMR-related energy expenditures is yet to be understood.

A second member of the AHSG family, AHSG-B, was identified recently (Olivier, E. et al., Biochem J 350, 589-597 (2000)). Whether AHSG-B shares irstatin activity with AHSG-A and/or whether such AHSG redundancy could protect against the deleterious effects of gene deletion is not known. Interestingly, mice deficient in PTP-1B demonstrate a phenotype similar to Ahsg KO mice, e.g., increased insulin sensitivity and IR phosphorylation, decreased adiposity and resistance to weight gain (Elchebly, M. et al., supra; Klaman, L. D. et al., supra). This was not unexpected since both AHSG and PTP-1B decrease IR phosphorylation. Further, AHSG KO mice demonstrate a phenotype in contrast to MIRKO (muscle-specific insulin receptor knockout) mice, which show peripheral insulin resistance with decreased IR, IRS-1 phosphorylation and glucose uptake in muscle with elevated fat mass, plasma triglyceride and FFA, but normal blood glucose, insulin and GTT (Bruning, J. C. et al., Mol Cell 2, 559-569 (1998)).

The following model for the role of Ahsg in the maintenance of glucose homeostasis is proposed (FIG. 11). The postprandial “sink” for blood glucose is chiefly skeletal muscle, due to its mass and density of GLUT4 glucose transporters relative to adipose tissue. During the first 2 h after a glucose challenge, the vast majority of glucose ends up in the glycogen stores of skeletal muscle (Shulman, G. et al., N Engl J Med 322, 223-228 (1990)). In the WT mouse, sufficient for AHSG, AHSG blunts insulin action on skeletal muscle, curtailing the function of muscle IR, thus dampening the size of the glycogen store and the rate at which glucose enters skeletal muscle. In the WT mouse, AHSG may act to spare some blood glucose for consumption by adipose tissue, a rather “sluggish” competitor for glucose. In contrast, the KO mouse shows hypersensitive skeletal muscle IR, enabling skeletal muscle to be an even better competitor for blood glucose than in the WT mouse. The KO mouse thus leaves little spare glucose for the “sluggish” adipose tissue, resulting in decreased adiposity and enhanced glycogen content of skeletal muscle.

In summary, this study provides the first direct evidence that AHSG has a critical role in clearance/uptake of glucose from blood and in modulating insulin sensitivity. Control of whole body glucose utilization by AHSG is probably mediated by modulation of the phosphorylation status of IR and downstream signaling proteins. Ahsg KO mice demonstrate lower plasma concentrations of free fatty acids and triglycerides, decreased adiposity, resistance to weight gain and remain insulin-sensitive on a high-fat diet. Taken together, these findings suggest a critical role for AHSG in regulating insulin action and lipid metabolism. Since AHSG is known to bind directly to activated IR, a pharmacological agent that interferes with AHSG binding to muscle IR or AHSG's ability to blunt IR function might provide a phenocopy of the KO mouse, with improved insulin sensitivity, decreased adiposity on normal diets, and resistance to weight-gain in HF diets.

### EXAMPLE VIII

### ELISA for Plasma ASHG

The inventors developed a sensitive and specific ELISA using commercially available polyclonal anti-AHSG antibodies. Using this assay, the concentration of plasma AHSG was investigated.

Immulon 1 plates (Dynatech Laboratories, Chantilly Va., USA), in a 96-well format, were coated with 2 μg/mL of AHSG (Calbiochem, La Jolla Calif., USA) in 0.1 mmol/L carbonate bicarbonate buffer, pH 9.6. After overnight incubation at 4° C., unbound material was removed by washing the plate three times with PBS/0.05% Tween-20. Uncoated sites were blocked with 1% BSA inPBS. AHSG standards in the range of 200-700 ng/nL or plasma dilutions ((1:750, 1:1000 or 1:2000) in phosphate buffered saline containing 0 1% BSA were incubated with commercial goat anti-human AHSG antibody (Incstar, Stillwater Minn., USA) at room temperature for 1.5 hrs and 75 μL of standard or dilution of patient's plasma was added to the wells and left overnight at 4° C. in the dark. ELISA plates were washed 3 times in PBS/0.05% Tween-20 and incubated with 75 μL swine anti-goat IgG conjugated with alkaline phosphatase (Caltag Laboratories, Burlingame Calif.) for 2 h at room temperature. The plates were washed again and 100 μL of p-nitrophenyl phosphate substrate (Chemicon, Temecula Calif., USA) was added and absorbance was read in an ELISA plate reader (Bio-Tek Instruments Inc, Burlington Vt., USA) at 405 nm after stopping the reaction with 100 μl of 3N NaOH.

Assay Evaluation

The analytical performance characteristics of the modified ELISA for measuring plasma AHSG concentrations were evaluated by determination of minimum detectable concentration (MDC) and assay precision. MDC was defined by the standard deviation (SD) (n=6 assays in quadruplicate) of dose measurement at zero-dose or background. The detection limit was calculated by the SD of zero-dose or background divided by the slope of the regression line.

Assay precision was determined by calculating the intra- (n=14 replicates) and inter-assay (n=6 runs, each in quadruplicate) coefficients of variation (CV %).

Analytical Methods

Plasma glucose was measured using Glucose Flex™ reagent cartridge on a Dimension®clinical chemistry system (Dade Behring Inc., Newark Del., USA) and insulin was enzyme immunoassay technique. Insulin resistance was assessed using a simple index as described by Duncan et.al. (Duncan M H et al., Lancet 1995;346:20-21). Briefly, the insulin resistance index (IRI) was obtained from the glucose concentration multiplied by the insulin concentration and divided by the normalized product of 5 mmol/L glucose and 5 munits/L insulin.

Results

ELISA for AHSG: Validation of Assay

In the process of developing a specific and sensitive ELISA for AHSG, several immunoassay formats including two-antibody sandwich assay, antigen capture assay and antibody capture assay using jacalin or purified AHSG (Calbiochem), were tested. Commercially available anti-AHSG polyclonal antibodies (Rinding Site, Birmingham, UK and Incstar Corporation) and several batches of polyclonal AHSG antibodies, generated in rabbits in the inventors' laboratory, were tested for specificity and sensitivity. The antibody capture assay using purified AHSG (Calbiochem) and anti-AHSG antibody (Incstar) was selected as the method of choice. This assay demonstrated excellent specificity with a high signal to background ratio compared to other ELISA formats. The specificity was tested using a bovine serum albumin standard. A typical standard curve generated with a purified AHSG standard was obtained. Plasma sample dilutions of 1:750, 1:1000 or 1:2000 produced concordant results (321.4±6.73 mg/L, n=8; 314.2±89 mg/L, n=8 and 327.8±5.0 mg/L, n=8, respectively). However, quantitation of data at higher plasma dilutions (1:6000 or 1:15,000) was inaccurate and therefore, plasma samples were diluted either to 1:750, 1:1000 or 1:2000 for all assays. The minimum detectable concentration of the assay was approximately 30 mg/L, as defined by the standard deviation of dose measurements at zero-dose. Typically, the intra-assay CV % was 2.5% at a concentration of 300.5 mg/L and the inter-assay CV % was 5.04% at a concentration of 311.2 mg/L

Plasma AHSG Concentration in Healthy Controls

AHSG concentrations, assayed by ELISA, in plasma samples from 44 apparently healthy individuals range from 210 to 450 mg/L, with a mean±SEM of 312.3±9.9 mg/L and a median of 305.5 mg/L. The 95% confidence intervals were 292.3 mg/L to 332.3 mg/L. PlasmaAHSG concentrations were not significantly different in men and women.

Plasma AHSG Concentration in Patients with Acute Myocardial Infarction (AMI)

Patients diagnosed with AMI tended to have a lower level of plasma AHSG at the time of admission, with a mean±SEM of 2813±25.8 mg/L compared to 3123±9.9 mg/L in healthy controls, though the differences were not statistically significant (F=0.142). AHSG concentrations ranged from 132-489 mg/L in AMI patients with a median of 248 compared to a median of 305.5 mg/L in the healthy control group. Forty percent of AMI patients showed AHSG concentrations below 200 mg/L compared to none in the healthy control group. It is notable that for AMI patients, the plasma AHSG concentrations were considerably more heterodisperse than for normals. During the recovery period, AHSG levels begin to increase, with a mean±SEM of 290.1±22.1 mg/L and a median of 280.5 mg/L. Though 25% of our AMI patients showed AHSG concentrations below 200 mg/L, a regression analysis comparing AHSG levels at the time of admission versus discharge showed a significant increase in matched-pair patient samples (r=0.45, p<0.01) (FIG. 3). In the follow-up phase, AHSG concentrations ranged from 228-431 mg/L, with a mean ! SEM of 340.8 0!0 339 mg/L and a median of 331 mg/L.

Correlation of AHSG with Insulin Concentrations

Plasma glucose and insulin concentrations are significantly elevated in patients diagnosed with AMI compared to healthy control (F<0.001). On discharge, plasma glucose and insulin levels are decreased significantly (F<0.01 and F<005, respectively) compared to concentrations on admission. However, compared to healthy control, plasma insulin levels remained significantly elevated on discharge and follow-up. The admission insulin-resistance index (AIRI) was significantly higher in samples from the AMI group compared to healthy controls (F<0.001). Blood sampling at discharge showed a significant decrease in IRI compared to AIRI (F<0.05) and remained unchanged on follow-up. Plasma AHSG concentrations demonstrated a significant negative correlation with levels of insulin in the AMI-admission group (r=−044, F<0.05). However, AIRI was not correlated with AHSG concentrations on admission (r=−0.35, p=0.126).

AHSG concentrations in plasma have traditionally been assayed by electro-immunodiffusion or rocket immunoelectrophoresis techniques. More recently, Akhoundi et al, reported development of an ELISA for quantitation of plasma AHSG, using antibodies generated in their laboratory (Akhoundi C et al., J Immunol Methods 1994; 172:189-196). However, use of their assay is limited because their antibodies are not commercially available. Therefore, to assay AHSG concentrations, the present inventors developed an ELISA, using commercially available antibodies. The “normal” reference range of plasma AHSG concentrations in the healthy control population was 292-332 mg/L The high specificity, high signal-to-background ratio, and the low inter- and intra-assay coefficient of variation (2-4%) of our assay validate its precision and reliability.

Since AMI has long been known to produce an inflammatory response and AHSG is a negative acute phase protein, decreased levels of plasma AHSG were anticipated in patients with AMI. The results confirm this hypothesis with lower plasma concentrations of AHSG (less than 200 mg/L in 40% of patients) in AMI patients on admission. In comparison, plasma AHSG concentrations were above 200 mg/L in all samples from individuals in the healthy control group. However, when grouped together, the differences between the admission AHSG concentrations in the AMI and healthy control groups were not statistically significant. This was not unexpected, as the duration and frequency of myocardial ischemic episodes regulates the acute phase response in patients with AMI.

The references cited above are all incorporated by reference herein, whether specifically incorporated or not.

Having now fully described this invention, it will be appreciated by those skilled in the art that the same can be performed within a wide range of equivalent parameters, concentrations, and conditions without departing from the spirit and scope of the invention and without undue experimentation.

1. A method for inhibiting the biological activity of α2-Heremans Schmid Glycoprotein (AHSG) protein in a cell comprising providing to the cell a compound that inhibits the phosphorylation of AHSG at one or both of Ser-120 and Ser-312 or dephosphorylates one or both of Ser-120 and Ser-312.

2. The method of claim 1 wherein the biological activity being inhibited is the binding of AHSG to muscle insulin receptor or the diminution of insulin receptor function.

3. The method of claim 1, wherein the inhibiting is achieved by contacting the cell with one or a combination of:
(a) a protein serine-threonine kinase inhibitor; and
(b) a serine phosphatase or a compound that induces or enhances the activity of the phosphatase.

4.-5. (canceled)

6. A method of augmenting the phosphorylation of, or tyrosine kinase activity of, insulin receptors in a liver or muscle cell, comprising providing to the cell a compound that lowers the amount of active AHSG or inhibits the biological activity of AHSG in the cell, thereby augmenting the phosphorylation and/or the tyrosine kinase activity.

7. The method of claim 6 wherein the augmenting is achieved by delivering to the cell an effective amount of an antisense nucleic acid construct that hybridizes with a sequence present in AHSG genomic DNA or with a coding nucleic acid sequence that encodes AHSG, thereby lowering the amount or inhibiting the activity of AHSG in the subject.

8. The method of claim 7 wherein the genomic DNA has the sequence SEQ ID NO:1 or wherein the coding sequence encodes a protein having a sequence selected from the group consisting of SEQ ID NO:2, SEQ ID NO:3, SEQ ID NO:5, SEQ ID NO:6 or SEQ ID NO:7.

9. (canceled)

10. The method of claim 8 wherein the coding sequence encodes a protein of SEQ ID NO:2 or SEQ ID NO:3.

11. The method of claim 6 wherein the compound is one or a combination of
(a) a serine-threonine kinase inhibitor that inhibits the phosphorylation of AHSG at one or both of Ser-120 and Ser-312 or
(b) a protein serine phosphatase or a compound that induces or enhances the activity of the phosphatase that dephosphorylates one or both of Ser-120 and Ser-312.

12. (canceled)

13. A method for treating a subject who is susceptible to, or suffers from, obesity and insulin resistance comprising lowering the amount of active AHSG or inhibiting the biological activity of AHSG in the subject.

14. The method of claim 13 wherein the lowering or the inhibiting is in liver or muscle.

15. The method of claim 13 wherein the inhibiting is achieved by delivering to the subject an effective amount of an antisense nucleic acid construct that hybridizes with a sequence present in AHSG genomic DNA or with a coding nucleic acid sequence that encodes AHSG, thereby lowering the amount or inhibiting the activity of AHSG in the subject.

16. The method of claim 15 wherein
(a) the genomic DNA has the sequence SEQ ID NO:1; and/or
(b) the antisense nucleic acid has between about 6 and about 30 nucleotides.

17. (canceled)

18. The method of claim 15 wherein the antisense construct is antisense to a sequence that includes the initiation codon of the AHSG.

19. The method of claim 16 wherein the antisense construct is antisense to a sequence that is part or all of an intron of SEQ ID NO:1.

20. The method of claim 15 wherein the coding sequence encodes a protein having a sequence selected from the group consisting of SEQ ID NO:2, SEQ ID NO:3, SEQ ID NO:5, SEQ ID NO:6 or SEQ ID NO:7.

21. The method of claim 20 wherein the coding sequence encodes a protein of SEQ ID NO:2 or SEQ ID NO:3.

22. The method of claim 13 wherein the inhibiting is achieved by administering to the subject an effective amount of an antibody specific for AHSG, whereby the antibody lowers the amount of or inhibits the biological activity of AHSG.

23. The method of claim 22 wherein the antibody is a monoclonal antibody.

24. The method of claim 22 wherein the subject is a human and the antibody is human or a humanized antibody.

25.-28. (canceled)


SEQUENCE LISTING (7 sequences)

SEQ ID NO: 1 - DNA, 8748 bp, Homo sapiens
gatcacagtagaagacatttcctctgctgccaaacccatggcactctgaggctgactgtgtccacctcattccctcagctgtcttctctttgctgctattaccatgttccaagcagactttggagcatctcccccacagcagcatggactttggcagatttcttggggaccagcgatgtcctaacctgtttgcttttccagggctgatgtttgcagggtgtttttttttttcttttgaaccaaagcagaaatcatcctgtatccttatgcaattcttccggcaggctccaacagataaataaagcccaccaccctccatgggtctacctttcccagcagagcacctgggttggtcccgaagcctccaaccacctgcacgcctgcctgccagggcctctctggggcagccatgaagtccctcgtcctgctcctttgtcttgctcagctctggggctggcactcagccccacatggcccagggctgatttatagacaaccgaactgcgatgatccagaaactgaggaagcagctctggtggctatagactacatcaatcaaaaccttccttggggatacaaacacaccttgaaccagattgatgaagtaaaggtgtggcctcaggtaagtggacctgctgtctatgagctgaaataatgtgtacatggagctcaatcaggtgcctcaaaaaatcaccatccacccagtgcaaatgaaaccacagaggagtaaattctctgatttcttcccaggagtgagggaaggggcaggcagagggcaggagaggagacattctgtatggcagtcatgggtgtcaggagggagctgggtggggtgtgaggtggtgtgcaggagaaaaagtgcttcaaatggtagtgtgcagatcacagacagaaagtgtaacttgctggaaaaactaggacccaagagaccagctcctagttgccaagttaccactggctgaaaatcacgtatctgtctttggtttggtttctctctaacaaagactgagaatgaataaaactagcatctggcagatgcctactatatgccaggcccattcacatagattatctcatttactctttcccggtcctgcctcctggtgctgtgtggtacatatattgttcttgtcttacccaagaggagaccaaggctctcttgtgtgtgtgtgtgcagttttttggtttttggttttgtttttttttttttggtccaaaatcatataattactaagtcttcaggctgggatttgattccatatctgtgttccaacttctacacaaactgcctcccaaagagagttacccacatcccagagagaagtcttggcataaacacaattcacctcctcacacactagacaggaaaccaacgcagcttgaagccagtgacaagaaaaatcaagctggaaatatgcctcggggatcagtcaagagatttggagaggtggaaagaagctgtctgcctactgcctgttttgaaattagatttatttctgattaaggacaattctttcagcaaatatgtattacaagccttccttggacaagaaccagagatattaggttgaaccatataaaactgccatttttctatatcaaaagcaaccaaatattggccgttttaatggttcaacctaatacagtggtgaaaaaggcacaatatgtgcccacaagagcttacaatctaggttggaaaataaggttcaacaacaggaagcctggaccgactgacgactgccatccgtctcacaaagagacaaaatatttgaaatcaggattgctccggatggattttaagagtgctgcagccatattaaagcacagtggtggttaggaggaaacgctgatcaagtcaggggaaatgaacacgcaacacgcacatctgagggaaaaggtaatcatgaatgggcattgtgacttttactaaaggcagagcttcagagttggttcccttgagaaacccaggtgtacccggttcctgttcgccagagctgtgaacgctttcaggcagtcactctgggcacacctggacatcataaaatgcggaacttctcccaggggaggggatgctgaggcttcaggtactagtgaatcaggcagaaccaatgagaggcaaacagagctgggctgagaggagaaaaggcatacttgtaccttctggtttttcaggttcgaagacaagatacagaaacaggtgaactcacaagaatatctccaaggattgttgcaagctccctcgtgtctacactagtgacatccagtttcctgtcagagggagacatgcccttccccattatcgccagcagggggaagtagagagcagcatcgttgcatgccggcacctgctgcacaagccaagacaaaggaaaaaccaaggacaacagcagcaaaaacctctaggagggaaaagaaaacggaggaaggaaggaaagcaaataatgaaaaggaagaaagaaagaaggagagggagggatagaggagattaaaaggccacagtaagatattaccctacaccacctattttgcagcttgtctgagaaaaatccaaacttgcattttcccaaagcactgcttgccgagtgaaatcttaaaaaataaaataaataataaatacaaataagtgttaacacccatttgtagttttcaaatagagcgcagagtgagggctgtggctccatcgacttgttcaagcccaggaccccgtctgctttgcgagcatcatctggtgcttccttaatcaacagacgaagaccagacaagccctggtcattgtcctgcccacaggccagttcagagctagacggagttgcagactgacagtaagaatgacatttccctcacctctccaaaagcggggtgctctcaagcccaatgagggcgcataccgtggaccgcaccacaggatcaggggaataggttgctcgcggcttcactctttgtctccacagcagccctccggagagctgtttgagattgaaatagacaccctggaaaccacctgccatgtgctggaccccacccctgtggcaagatgcagcgtgaggcagctgaaggagcatgtgagtacccttcttaggatgactgtaggtggcccttcggccagctccaccgattcacccagcgtctcagcctgccttcttggctagccagggtgcagtttctaaaattgccatttgtggccgagcgcagtggctcatgcctataatctcagcactttgggaggctgaggcaagtggatcgcctgaggtcaggagttcaagaccagcctggccagtatggtgaaaccccatctctactaaaaatacaaaaattagctggacgtggtgacgggcacctgtaaatcccagctcctcgggaggctgaggcaggagaatcgcttgaacccgggaggtggaggttgcagtgagccaagatcctgccattgcactccagcctgggcaacaacagtgaatctctatctcgaaataataataataatcatcatcatcataaataaaattgccatttgatgccacttgccctggggctgagttttacaagcgtttaactatatcgttgtatccctgaaagctgagagtgccatgtttcagtattacccagcaaaggcgattttgcaagggtcacctttgacagccgtgcctggagggagcctgcccggggtgcgaaggggaagggcagccatcctcacgtgggtttctttctccaggctgtcgaaggagactgtgatttccagctgttgaaactagatggcaagttttccgtggtatacgcaaaatgtgattccagtccaggtacagatgactattcttattctcattttttccttgtagagaaagtggggaagggatctgaataattttcaacttaagtagttctagcagctttgtcggtgaggaaaaggagaagccaaatttcctgggttctgggatttttaaaattgtgttttaagaagctactcttggcctggtgcggtggctcacgcctgtaatccacccacccgaggcaggtggatcacctgaagtcagaagttcgagaccagcctggccaacatagtgaaacccccatctctactaaaaatacaaaaatgtggtggtgctcgcctgtaatcccagctactagggaggctgaggcaggagaatcgcttgaacctgggaggcagaggtggcagtgggccgagatcgcaccactgcactccagcctgagtgacacagagtgagaccctgtctcccaaaaataagaagttattcttactggaagtgaaaattgcctcgtgatgataagagctccttcagaaatgtcagcatagccaaagccttttgaaggtttagtaagaagcagagaaagtgcctgaagctatctggggaatgccttagcccttgctaacgcagcagagctggggccatgccagggagaatggctgcccacatcctggtttcctctctccgagcagactcagccgaggacgtgcgcaaggtgtgccaagactgccccctgctggccccgctgaacgacaccagggtggtgcacgccgcgaaagctgccctggccgccttcaacgctcagaacaacggctccaattttcagctggaggaaatttcccgggctcagcttgtggtaaagactgagattcttttgacaggttgggcagttcggtggcacttcgggaatgtactgtacgtggtggagcgggaggcagggcaagaacaggcgcaggggcagcgatgagaaagcaaggagagggttgtttggaaagggaagaaagcatcctaagggggtatgaggctcctgagtgtcatgaggaccccaacaccctcagcgcctcccccatgctgagccactgtaacgtccagcagccacagctgccggcaggtacatccccactccctccgttccagctaaaaccaaagctcagtgtcagctggtagagtttgcccacgtcggccagaagcactcactgtaaatttgctgggctccagtaccacccatctccgctgaacatctgccacagactcgtaattaatactcacttgtgctgacaagcttataatggcaagatcttaaaatgcctttcgagtcactggagaaaacatctcattgtactgtgggtggtttagcacattggaattcaacagaattcaaatgtttaagaaaatgtattctggatatcagccatggccatacttggaaatacgctagtatagacggcaattctattaatcagaatatgtgattctcagaacatccccaccccagactacaccaaataacagatattttattgtgtccatatgctccaactactttaaaaaagaaaagctcaagtgatatcttccatactttcatctaaatcttttcatttgagcctgctctatgaaacaggtggaagaggtattaatctcttcactttcccaccctatcttggaataacctgaaccttgggtatcaagtgcagcccaagagtgagggctggggggaggcagggttcccactcctatcagtctaaggctggccttctgattccggtttcctatctggaaactcacctccaccctgaaggaccggtgatggaaactttcccctcctacaagggagacacaacccctacctctaaagcacaagcacttgagaacacaaccccataacaacttccctatgtaaaccattgagggacatgtcttctgggccgacgcatggtctgcatgaatggtgctccccgaaggaggctacttcccgctctccttctctgcccttttcattgtaagtcatctttcctcaagagcattttcatgtactcttctcagcccctcccaccttctacctatgtggagtttacagtgtgtggcactgactgtgttgctaaagaggccacagaggcagccaagtgtaacctgctggcagaaaaggtgagtgggccgggaccttggggtgttaccactcggacagagctgtttgtggaacagaacatccttggttagtttgtttcttggggctgcagacagagaataacagtgaaaatcccctctccctgtggatcacggaaagcctccttttagggtgtcacctcatccctttaagagctgtcatcaaatcatctcacccactggaagcacatgaagttaggagaaagagagaggttatttgttaatgaagccaagtcacgcccacccactgggaatgtgaagtgcacatttcctagacatataactctgatacaaaagctttcaagtccttgagccaataatgtacacttctaggatttcagtcttaagaagtcatcaagtggccaggcatgatggttcatgcctgtaatccagcactttgggaggccaagacgggtggatcgggaggtcaggagatcgagaccatcctggctaacatggtgaaaccccgtctctactaaaaatacaaaaaaattagccaggcttggtggtgagcgcctgtagtcccagctactcgggaggctgaggcaggagaatggtgtgaacccaggaggcagatgttgcagtaaactaagatcgtgccactgcactccagcctgggcaacagaacgagactctgtctcaagaaaaaaagaaaaagaaaaagaattcctccgtgacatttgacagaatatatctataaaaatgatttattatggatataaagagaccaaaaaagagagatctgtatgtccaacaggaaggtgtcattgaataatccatgcacatcagtaaatagaaaattgtgcagacactaaaaattgtgttttcaaggaataatgaatgatatgagaaaatgctattatggcaagtgaaaacacacaggatacaacatcgtatagtcacaatgatctcaatttttaaatcatatttaatagtattttaaaataagttagaaatgcatcaatgttaacagtccttctttctaggccaccaccagaaagggattatgggtaatctctctcactctccaagtatttctgtatttccatgttatatatagaatcatatacctcccacaagcagaaactataactttaagaaaaatggtttttccaactaatttaaggttggcgcgtcaatgaaattgggggggatccatttttgaaattagttaaaataaatcctctttctctgtgggcagcaatatggcttttgtaaggcaacactcagtgagaagcttggtggggcagaggttgcagtgacctgcacggtgttccaaacacaggtaacagctccgtgaatattcttgcctacaccttcagaatacaatgaccccttcacatttatgcagtgcagtagtgatgacaggacatttgctctcccgtgcttctgaatctcacagtatgaaataacactggggtatgcggaatcatcaacaaatggaaggatattttagctatgcctttccctcccacgaactagtgacatacgggaagaaccatcttactgtgtagttgacaaagccacctttttatttgtgggaggtgggagtggttttctgagttgcagagaccaggtggccagatctacctgttagctcccagtggctgcagcttcagatgacaaagagggtggcactgctgggcaagggtgagccataggtggggtgcttttactcattggacatatgtgtgtaagtccaccatcacaaagacaatcctagtgaggccggggcaacataggccagtcacccctccttgtaaccttgatgacaatcccttgtacttaggtaggtcctttcttgctagactctttgcaaataaaaatgtataatgtgaggaaattgggtgccagtgccacctgggcctgtgggttgtcttgcctgggaggaggaagcaaactaactgaaggaaatggtcctttttccagcccgtgacctcacagccccaaccagaaggtgccaatgaagcagtccccacccccgtggtggacccagatgcacctccgtcccctccacttggcgcacctggactccctccagctggctcacccccagactcccatgtgttactggcagctcctccaggacaccagttgcaccgggcgcactacgacctgcgccacaccttcatgggtgtggtctcattggggtcaccctcaggagaagtgtcgcacccccggaaaacacgcacagtggtgcagcctagtgttggtgctgctgctgggccagtggttcctccatgtccggggaggatcagacacttcaaggtctaggctagacatggcagagatgaggaggtttggcacagaaaacatagccaccattttgtccaagcctgggcatgggtggggggccttgtctgctggccacgcaagtgtcacatgcgatctacattaatatcaagtcttgactccctacttcccgtcattcctcacaggacagaagcagagtgggtggtggttatgtttgacagaaggcattaggttgacaacttgtcatgattttgacggtaagccaccatgattgtgttctctgcctctggttgaccttacaaaaaccattggaactgtgactttgaaaggtgctcttgctaagcttatatgtgcctgttaatgaaagtgcctgaaagaccttccttaataaagaaggttctaagctgaatgtggtcatgcttattgcgacttcatcccagctcccctcacatgcatagccttttaccccaacaaacacagtgtccctaatcaaaaccaaagtgaaaagagaaccaaaagagaacaaaaacctgctgtattgccagatacaggaaaaagtgagactaggatc

SEQ ID NO: 2 - protein, 367 aa, Homo sapiens [sequence omitted]

SEQ ID NO: 3 - protein, 367 aa, Homo sapiens
MKSLVLLLCLAQLWGCHSAPHGPGLIYRQPNCDDPETEEAALVAIDYINQNLPWGYKHTLNQIDEVKVWPQQPSGELFEIEIDTLETTCHVLDPTPVARCSVRQLKEHAVEGDCDFQLLKLDGKFSVVYAKCDSSPDSAEDVRKVCQDCPLLAPLNDTRVVHAAKAALAAFNAQNNGSNFQLEEISRAQLVPLPPSTYVEFTVSGTDCVAKEATEAAKCNLLAEKQYGFCKATLSEKLGGAEVAVTCMVFQTQPVSSQPQPEGANEAVPTPVVDPDAPPSPPLGAPGLPPAGSPPDSHVLLAAPPGHQLHRAHYDLRHTFMGVVSLGSPSGEVSHPRKTRTVVQPSVGAAAGPVVPPCPGRIRHFKV

SEQ ID NO: 4 - protein, 398 aa, Homo sapiens
MKSLVLLLCLAQLWGCHSAPHGPGLIYRQPNCDDPETEEAALVAIDYINQNLPWGYKHTLNQIDEVKVWPQQPSGELFEIEIDTLETTCHVLDPTPVARCSVRQLKEHAVEGDCDFQLLKLDGKFSVVYAKCDSSPDSAEDVRKVCQDCPLLAPLNDTRVVHAAKAALAAFNAQNNGSNFQLEEISRAQLVPLPPSTYVEFTVSGTDCVAKEATEAAKCNLLAEKQYGFCKATLSEKLGGAEVAVTCMVFQTQPVSSQPQPEGANEAVPTPVVDPDAPPSPPLGAPGLPPAGSPPDSHVLLAAPPGHQLHRAHYDLRHTFMGVVSLGSPSGEVSHPRKTRTVVQPSVGAAAGPVVPPCPGRIRHFKVSLEGPRFEGKPIPNPLLGLDSTRTGHHHHHH

SEQ ID NO: 5 - protein, 346 aa, Murinae gen. sp.
MKSLVLLLCFAQLWGCQSAPQGTGLGFRELACDDPEAEQVALLAVDYLNNHLLQGFKQVLNQIDKVKVWSRQRPFGVVYEMEVDTLETTCHALDPTPLANCSVRQLTEHAVEGDCDFHILKQDGQFRVMHTQCHSTPDSAEDVRKLCPRCPLLTPFNDTNVVHTVNTALAAFNTQNNGTYFKLVEISRAQNVPLPVSTLVEFVIAATDCTAKEVTDPAKCNLLAEKQHGFCKANLMHNLGGEEVSVACKLFQTQPQPANANAVGPVPTANAALPADPPASVVVGPVVVPRGLSDHRTYHDLRHAFSPVASVESASGETLHSPKVGQPGAAGPVSPMCPGRIRHFKI

SEQ ID NO: 6 - protein, 352 aa, Rattus norvegicus
MKSLVLLLCFAQLWSCQSAPQGAGLGFRELACDDPETEHVALIAVHYLNKHLLQGFRQILNQIDKVKVWSRRPFGQVYELEIDTLETTCHALDPTPLANCSVRQQAEHAVEGDCDFHILKQDGQFRVLHAQCHSTPDSAEDVRKFCPRCPILIRFNDTNVVHTVKTALAAFNAQNNGTYFKLVEISRAQNVPFPVSTLVEFVIAATDCTGQEVTDPAKCNLLAEKQYGFCKATLIHRLGGEEVSVACKLFQTQPQPANANPAGPAPTVGQAAPVAPPAGPPESVVVGPVAVPLGLPDHRTHHDLRHAFSPVASVESASGEVLHSPKVGQPGDAGAAGPVAPLCPGRVRYFKI

SEQ ID NO: 7 - protein, 359 aa, Bos taurus
MKSFVLLFCLAQLWGCHSIPLDPVAGYKEPACDDPDTEQAALAAVDYINKHLPRGYKHTLNQIDSVKVWPRRPTGEVYDIEIDTLETTCHVLDPTPLANCSVRQQTQHAVEGDCDIHVLKQDGQFSVLFTKCDSSPDSAEDVRKLCPDCPLLAPLNDSRVVHAVEVALATFNAESNGSYLQLVEISRAQFVPLPVSVSVEFAVAATDCIAKEVVDPTKCNLLAEKQYGFCKGSVIQKALGGEDVRVTCTLFQTQPVIPQPQPDGAEAEAPSAVPDAAGPTPSAAGPPVASVVVGPSVVAVPLPLHRAHYDLRHTFSGVASVESSSGEAFHVGKTPIVGQPSIPGGPVRLCPGRIRYFKI